SEQUENCE LISTING (19 sequences)

SEQ ID NO: 1 - DNA, 1640 bp, Artificial
  Engineered mouse Akt1 comprising three NLS
cagtgcgatcgcggcgcgccgccaccatgaacgacgtagccattgtgaaggagggctggctgcacaaacgaggggaatatattaaaacctggcggccacgctacttcctcctcaagaacgatggcacctttattggctacaaggaacggcctcaggatgtggatcagcgagagtccccactcaacaacttctcagtggcacaatgccagctgatgaagacagagcggccaaggcccaacacctttatcatccgctgcctgcagtggaccacagtcattgagcgcaccttccatgtggaaacgcctgaggagcgggaagaatgggccaccgccattcagactgtggcagatggactcaagaggcaggaagaagagacgatggacttccgatcaggctcacccagtgacaactcaggggctgaagagatggaggtgtccctggccaagcccaagcaccgtgtgaccatgaacgagtttgagtacctgaagctactgggcaagggcacctttgggaaggtgattctggtgaaagagaaggccacaggccgctactatgccatgaagatcctcaagaaggaggtcatcgtcgccaaggatgaggttgcccacacgcttactgagaaccgtgtcctgcagaactctaggcatcccttccttacggccctcaagtactcattccagacccacgaccgcctctgctttgtcatggagtatgccaacgggggcgagctcttcttccacctgtctcgagagcgtgtgttctccgaggaccgggcccgcttctatggtgcggagattgtgtctgccctggactacttgcactccgagaagaacgtggtgtaccgggacctgaagctggagaacctcatgctggacaaggacgggcacatcaagataacggacttcgggctgtgcaaggaggggatcaaggacggtgccactatgaagacattctgcggaacgccggagtacctggcccctgaggtgctggaggacaacgactacggccgtgcagtggactggtgggggctgggcgtggtcatgtacgagatgatgtgtggccgcctgcccttctacaaccaggaccacgagaagctgttcgagctgatcctcatggaggagatccgcttcccgcgcacactcggccctgaggccaagtccctgctctccgggctgctcaagaaggaccctacacagaggctcggtgggggctccgaggatgccaaggagatcatgcagcaccggttctttgccaacatcgtgtggcaggatgtgtatgagaagaagctgagcccacctttcaagccccaggtcacctctgagactgacaccaggtatttcgatgaggagttcacagctcagatgatcaccatcacgccgcctgatcaagatgacagcatggagtgtgtggacagtgagcggaggccgcacttcccccagttctcctactcagccagtggcacagccgcggccgcagatccaaaaaagaagagaaaggtagatccaaaaaagaagagaaaggtagatccaaaaaagaagagaaaggtagatacggccgcagaacaaatggactacaaagacgatgacgataaaatggactacaaagacgatgacgataaatgaggcgcgccacgcgtcagt

SEQ ID NO: 2 - DNA, 9 bp, Artificial
  Linker1
gcggccgca

SEQ ID NO: 3 - skipped

SEQ ID NO: 4 - DNA, 18 bp, Artificial
  Linker2
gatacggccgcagaacaa

SEQ ID NO: 5 - protein, 480 aa, Homo sapiens [sequence omitted]

SEQ ID NO: 6 - DNA, 3008 bp, Homo sapiens
taattatgggtctgtaaccaccctggactgggtgctcctcactgacggacttgtctgaacctctctttgtctccagcgcccagcactgggcctggcaaaacctgagacgcccggtacatgttggccaaatgaatgaaccagattcagaccggcaggggcgctgtggtttaggaggggcctggggtttctcccaggaggtttttgggcttgcgctggagggctctggactcccgtttgcgccagtggcctgcatcctggtcctgtcttcctcatgtttgaatttctttgctttcctagtctggggagcagggaggagccctgtgccctgtcccaggatccatgggtaggaacaccatggacagggagagcaaacggggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 7 - protein, 480 aa, Mus musculus
MNDVAIVKEGWLHKRGEYIKTWRPRYFLLKNDGTFIGYKERPQDVDQRESPLNNFSVAQCQLMKTERPRPNTFIIRCLQWTTVIERTFHVETPEEREEWATAIQTVADGLKRQEEETMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPTQRLGGGSEDAKEIMQHRFFANIVWQDVYEKKLSPPFKPQVTSETDTRYFDEEFTAQMITITPPDQDDSMECVDSERRPHFPQFSYSASGTA

SEQ ID NO: 8 - DNA, 2707 bp, Mus musculus
gcggggcggggagaaggcgggccggcggcggcggcggcagcaccgagtcggcgggcggccggcccagcgcggcagcgcacgcgagtccgggaccagcggagcggaccgagcagcgtcctgtggccggcaccgcggcggcccagatccggccagcagcgcgcgcccggacgccgctgccttcagccggccccgcccagcgcccgcccgcgggatgcggagcggcgggcgcccgaggccgcggcccggctaggcccagtcgcccgcacgcggcggcccgacgctgcggccaggccggctgggctcagcctaccgagaagagactctgagcatcatccctgggttacccctgtctctgggggccacggataccatgaacgacgtagccattgtgaaggagggctggctgcacaaacgaggggaatatattaaaacctggcggccacgctacttcctcctcaagaacgatggcacctttattggctacaaggaacggcctcaggatgtggatcagcgagagtccccactcaacaacttctcagtggcacaatgccagctgatgaagacagagcggccaaggcccaacacctttatcatccgctgcctgcagtggaccacagtcattgagcgcaccttccatgtggaaacgcctgaggagcgggaagaatgggccaccgccattcagactgtggcagatggactcaagaggcaggaagaagagacgatggacttccgatcaggctcacccagtgacaactcaggggctgaagagatggaggtgtccctggccaagcccaagcaccgtgtgaccatgaacgagtttgagtacctgaagctactgggcaagggcacctttgggaaggtgattctggtgaaagagaaggccacaggccgctactatgccatgaagatcctcaagaaggaggtcatcgtcgccaaggatgaggttgcccacacgcttactgagaaccgtgtcctgcagaactctaggcatcccttccttacggccctcaagtactcattccagacccacgaccgcctctgctttgtcatggagtatgccaacgggggcgagctcttcttccacctgtctcgagagcgtgtgttctccgaggaccgggcccgcttctatggtgcggagattgtgtctgccctggactacttgcactccgagaagaacgtggtgtaccgggacctgaagctggagaacctcatgctggacaaggacgggcacatcaagataacggacttcgggctgtgcaaggaggggatcaaggacggtgccactatgaagacattctgcggaacgccggagtacctggcccctgaggtgctggaggacaacgactacggccgtgcagtggactggtgggggctgggcgtggtcatgtacgagatgatgtgtggccgcctgcccttctacaaccaggaccacgagaagctgttcgagctgatcctcatggaggagatccgcttcccgcgcacactcggccctgaggccaagtccctgctctccgggctgctcaagaaggaccctacacagaggctcggtgggggctccgaggatgccaaggagatcatgcagcaccggttctttgccaacatcgtgtggcaggatgtgtatgagaagaagctgagcccacctttcaagccccaggtcacctctgagactgacaccaggtatttcgatgaggagttcacagctcagatgatcaccatcacgccgcctgatcaagatgacagcatggagtgtgtggacagtgagcggaggccgcacttcccccagttctcctactcagccagtggcacagcctgaggcctggggcagcggctggcagctccacgctcctctgcattgccgagtccagaagccccgcatggatcatctgaacctgatgttttgtttctcggatgcgctggggaggaaccttgccagcctccaggaccaggggaggatgtttctactgtgggcagcagcctacctcccagccaggtcaggaggaaaactatcctggggtttttcttaatttatttcatccagtttgagaccacacatgtggcctcagtgcccagaacaattagattcatgtagaaaactattaaggactgacgcgaccatgtgcaatgtgggctcatgggtctgggtgggtcccgtcactgcccccattggcctgtccaccctggccgccacctgtctctagggtccagggccaaagtccagcaagaaggcaccagaagcacccccctgtggtatgctaactggccctctccctctgggcggggagaggtcacagctgcttcagccctagggctggatgggatggccagggctcaagtgaggttgacagaggaacaagaatccagtttgttgctgtgtcccatgctgttcagagacatttaggggattttaatcttggtgacaggagagcccctgccctcccgcacccgctcccgcgtggtggctcttagcgggtaccctgggagcgcctgcctcacgtgagcccttctcctagcacttgtccttttagatgctttccctctcccgctgtccgtcaccctggcctgtcccctcccggccagacgctggccattgctgcaccatgtcgttttttacaacattcagcttcagcatttttactattataataagaaactgtccctccaaattcaataaaaattgcttttcaagcttgaaaaaaaaaaaaaaaaa

SEQ ID NO: 9 - protein, 7 aa, Artificial
  SV40 large T antigen NLS
PKKKRRV

SEQ ID NO: 10 - protein, 16 aa, Artificial
  nucleoplasmin NLS
KRPAATKKAGQAKKKK

SEQ ID NO: 11 - protein, 8 aa, Artificial
  NLS
DPKKKRKV

SEQ ID NO: 12 - DNA, 46 bp, Artificial
  Primer FW
cagtgcgatcgcggcgcgccgccaccatgaacgacgtagccattgt

SEQ ID NO: 13 - DNA, 93 bp, Artificial
  Primer REV
actgacgcgtggcgcgcctcatttatcgtcatcgtctttgtagtccattttatcgtcatcgtctttgtagtccatttgttctgcggccgtatc

SEQ ID NO: 14 - DNA, 20 bp, Artificial
  AKT1seq-FW1
cgacgtagccattgtgaagg

SEQ ID NO: 15 - DNA, 20 bp, Artificial
  AKT1seq-REV1
acacctccatctcttcagcc

SEQ ID NO: 16 - DNA, 20 bp, Artificial
  AKT1seq-FW2
ggctgaagagatggaggtgt

SEQ ID NO: 17 - DNA, 20 bp, Artificial
  AKT1seq-REV2
cgttcttctcggagtgcaag

SEQ ID NO: 18 - DNA, 20 bp, Artificial
  AKT1seq-FW3
gctgctcaagaaggacccta

SEQ ID NO: 19 - DNA, 20 bp, Artificial
  AKT1seq-REV3
tagtccatttgttctgcggc

(19) United States
(12) Patent Application Publication
(10) Pub. No.: US 2022/0010289 A1
(43) Pub. Date: Jan. 13, 2022

(54) Materials and Methods for Pathologies in Muscle following Injury, Disease or Aging

(71) Applicant: The Brigham and Women`s Hospital, Inc., Boston, MA (US)

(72) Inventor: Ronald L. Neppl, Somerville, MA (US)

(21) Appl. No.: 17/294,031

(22) PCT Filed: Nov. 18, 2019
(86) PCT No.: PCT/US2019/061977

§ 371 Date: May 14, 2021

Related U.S. Application Data
(60) Provisional application No. 62/768,330, filed on Nov. 16, 2018.

(51) Int. Cl. C12N 9/12; A01K 67/027; A61P 21/00

(52) U.S. Cl. CPC C12N 9/12; A01K 67/0275; A61K 38/00; C07K 2319/00; A61P 21/00

(57) ABSTRACT

Compositions comprising nuclear-localised Akt1 (Akt-NLS) fusion proteins, and methods of use thereof in cellular and animal models, and for treating muscle pathologies.

### CLAIM OF PRIORITY

This application claims the benefit of U.S. Provisional Application Ser. No. 62/768,330, filed on Nov. 16, 2018. The entire contents of the foregoing are incorporated herein by reference.

### TECHNICAL FIELD

Provided herein are compositions comprising nuclear-localised Akt1 (Akt-NLS) fusion proteins, and methods of use thereof in cellular and animal models, and for treating muscle pathologies.

### BACKGROUND

Skeletal muscle has the remarkable ability to both modulate its mass and to regenerate damaged myofibers following injury. These processes are modulated by multiple factors including physical activity level, nutritional input, overall health, and genetic makeup. However with advancing age, skeletal muscle gradually loses mass, strength, and regenerative potential [1] while accumulating intramuscular adipose and fibrotic tissue [2, 3]. Though this progressive loss of muscle quality with advancing age is considered physiologically normal, in a subset of individuals, its increasingly rapid progression may lead to sarcopenia. The progressive nature of sarcopenia primarily arises from a multitude of aging-associated factors including the accumulation of macromolecular and cellular damages, hormonal changes, and increases in both oxidative stress and systemic inflammation [4, 5].

Skeletal muscle satellite cells (SCs) are a heterogenous population of quiescent muscle resident myogenic stem cells that reside in a niche between the basal lamina and the plasma membrane of the muscle fiber [6]. Following injury, SCs become activated, enter the cell cycle, and differentiate into committed mononuclear myocytes that ultimately fuse with existing myofibers to effect repair [7]. Though capable of self-renewal, the overall content [8] and myogenic potential [9] of SCs diminishes with age. Multiple parabiosis studies have indicated that circulating factors from young mice can improve the regenerative potential of old mice, and conversely, circulating factors from old mice impair the regenerative potential of young mice [10-13]. Consistent with these observations, increases in muscle derived FGF2 [14] and Wnt3a [10] have been shown to impair SC self-renewal and myogenic fate potential, respectively. Importantly, skeletal muscle is known to be a source of multiple circulating factors or ‘myokines’ including myostatin, IL-6, IL-7, IL-8, CXCL1, LIF, and others [15] known to promote myogenic repair [16-18].

### SUMMARY

Skeletal muscle regeneration following injury is a complex multi-stage process involving the recruitment of inflammatory cells, the activation of muscle resident fibroblasts, and the differentiation of activated myoblasts into myocytes. Dysregulation of these cellular processes is associated with ineffective myofiber repair and excessive deposition of extracellular matrix proteins leading to fibrosis. PI3K/Akt1 signaling is a critical integrator of intra- and intercellular signals connecting nutrient availability to cell survival and growth. Activation of the PI3K/Akt1 pathway in skeletal muscle leads to hypertrophic growth and a reversal of the changes in body composition associated with obesity and advanced age. Though the molecular mechanisms mediating these effects are incompletely understood, changes in paracrine signaling are thought to play a key role. Here, we utilized modified RNA to study the biological role of the transient translocation of Akt1 to the myonuclei of maturing myotubes. Using a conditioned medium model system, we show that ectopic myonuclear Akt1 suppresses fibrogenic paracrine signaling in response to oxidative stress, and that interventions that increase or restore myonuclear Akt1 may impair fibrosis.

Thus, provided herein are engineered proteins comprising an AKT serine/threonine kinase 1 (Akt1) linked to at least one nuclear localization sequence (NLS), optionally with a linker sequence therebetween. These proteins are sometimes referred to herein as Akt1-NLS proteins or fusion proteins. The proteins can also include one or more tag or purification sequences, e.g., 6× HIS or FLAG, as are known in the art.

In some embodiments, the Akt1 comprises mouse or human Akt1. In some embodiments, the mouse Akt1 sequence is at least 80% identical to SEQ ID NO:7. In some embodiments, the human Akt1 sequence is at least 80% identical to SEQ ID NO:5.

In some embodiments, the NLS comprises SV40 large T antigen NLS (PKKKRRV (SEQ ID NO:9)); nucleoplasmin NLS (KRPAATKKAGQAKKKK (SEQ ID NO:10); or DPKKKRKV (SEQ ID NO:11).

Also provided herein are isolated nucleic acids encoding the engineered Akt1-NLS proteins, and vectors comprising the nucleic acids, optionally along with regulatory sequences for expression of the Akt1-NLS proteins, e.g., a promoter. In some embodiments, the vector is a plasmid vector or viral vector. In some embodiments, the vector is an adeno-associated virus (AAV) vector, e.g., an AAV serotype-9 (AAV9).

In some embodiments, the isolated nucleic acid is a modified synthetic RNA, e.g., modified to include a 5′ cap and/or a 3′ polyadenylation sequence.

Also provided herein are isolated cells comprising the isolated Akt1-NLS nucleic acids, optionally expressing the engineered Akt1-NLS proteins described herein.

Additionally provided herein are transgenic animals, e.g., mice, wherein one or more cells of the animal (e.g., mouse) comprises a sequence encoding an AKT serine/threonine kinase 1 (Akt1) linked to at least one nuclear localization sequence (NLS), optionally with a linker sequence therebetween, integrated in to the genome of the cell. Also provided are isolated cells or tissues from the transgenic animals.

Additionally, provided herein are methods for reducing muscle fibrosis in a subject. The methods comprise administering to muscle tissue of the subject a protein comprising an AKT serine/threonine kinase 1 (Akt1) linked to at least one nuclear localization sequence (NLS), optionally with a linker sequence therebetween. Also provided are the proteins for use in methods for reducing muscle fibrosis in a subject.

Further, provided herein are methods for reducing muscle fibrosis in a subject. The methods include administering a nucleic acid encoding a protein comprising an AKT serine/threonine kinase 1 (Akt1) linked to at least one nuclear localization sequence (NLS), optionally with a linker sequence therebetween, preferably wherein the nucleic acid comprises a promoter that directs expression specifically in striated muscle cells of the subject. Also provided are the nucleic acids for use in methods for reducing muscle fibrosis in a subject.

In some embodiments, the promoter that directs expression specifically in skeletal muscle cells of the subject.

In some embodiments, the nucleic acid comprises (or is in) a viral vector. In some embodiments, the viral vector is an adeno-associated virus (AAV) vector, e.g., an AAV serotype-9 (AAV9).

In some embodiments, the nucleic acid comprises a modified synthetic RNA, e.g., modified to include a 5′ cap and/or polyadenylation sequence.

In some embodiments, the subject has muscular dystrophy, is a trauma patient who has experienced volumetric muscle loss; is a surgical patient in whom incisions through muscle fascia have resulted in extensive fibrosis; has a mitochondrial/metabolic myopathy; has an idiopathic inflammatory myopathy; has myocardial remodeling/hypertrophy/heart failure.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Akt1 is transiently localized in the nucleus during C2C12 differentiation. Western blot analysis of nuclear and cytoplasmic fractions obtained from differentiating C2C12 myoblasts. GAPDH is a used as a cytoplasmic marker while HnRNP A1 or Histone H3 serve as nuclear fraction marker. Data are representative of n=3 independent experiments.

FIGS. 2A-C. Synthetic modified RNA is an effective method for transient transfection in mature myotubes. (A) Immunofluorescence staining of C2C12 myotubes after transfection with either modGFP-NLS or a plasmid coding for GFP-NLS. Cells were fixed at the indicated times following transfection. Scale bar=50 μm. (B) Quantification of the percentage of GFP⁺ nuclei in MF20⁺ and MF20⁻ cells. (C) ModAkt1-NLS did not impair the expression of C2C12 differentiation markers. Data are normalized to DO controls and are representative of n=2 independent experiments.

FIGS. 3A-B. Ectopic myonuclear Akt1 impairs fibrogenic paracrine signaling. (A) Schematic of experimental procedures. (B) Quantitative PCR analysis of gene expression in 10T1/2 cells following 24-hour treatment with C2C12 conditioned medium. MC—unconditioned differentiation medium, CM—conditioned differentiation medium, H₂O₂—conditioned medium of C2C12 myotubes treated with H₂O₂ and modRNA encoding either Akt-NLS or GFP-NLS. Data are mean±standard deviation from n=3 independent experiments.

FIG. 4. Mouse validation performed in Akt-NLS and HSA-MCM; Akt-NLS mice. Mice at 2 months of age were treated with tamoxifen for 5 consecutive days for CRE activation. After 2 days of tamoxifen washout, mouse skeletal muscles were harvested and assay for transgene overexpression by western blot. Endogenous Akt1 was observed, as was the FLAG tagged transgenic Akt. Note that the transgene was only observed in the CRE+ mouse following tamoxifen injection.

FIG. 5. Pilot qPCR performed on gastrocnemius muscles. Gastrocnemius muscles were obtained from n=2 Akt-NLS and HSA-MCM; Akt-NLS mice following tamoxifen treatment described above.

FIG. 6. Reduced myonuclear Akt1 content in aged Mdx mice.

### DETAILED DESCRIPTION

Akt1 is a key integrator of intra- and intercellular signals known to promote growth and survival in multiple cell types [19, 20]. In skeletal muscle, IGF-1/PI3K/Akt1 signaling promotes hypertrophic growth by both activating anabolic cellular process via mTor and S6K1, while simultaneously inhibiting Foxo1/3 induced catabolic processes [20, 21]. Transgenic overexpression of IGF-1 [22], or a constitutively active form of Akt1 [23, 24] specifically in the skeletal muscle induces myofiber hypertrophy of both young and aged mice. Despite its lack of a conserved nuclear localization sequence [25, 26], Akt1 is known to translocate to the nucleus of multiple cancer cell lines [25, 27, 28]. In post-mitotic cardiomyocytes, Akt1 translocates into the nucleus following atrial natriuretic peptide stimulation promoting cardiomyocyte survival [29, 30]. However, the biological role of nuclear Akt in skeletal myofibers has not been explored.

In multiple cell types, the serine/threonine kinase Akt/PKB has emerged as a key integrator of intra- and intercellular signals that promote cell growth and survival. Acting downstream of IGF-1/PI3K, Akt1 signaling has a critical role in promoting skeletal muscle hypertrophy by activating anabolic processes via mTor and S6K1 while simultaneously inhibiting Foxo1/3 induced catabolic processes [20, 43]. Skeletal muscle specific transgenic overexpression of IGF-1 [22] or a constitutively active form of Akt1 [44] induces muscle hypertrophy, reversing the age [24] and high-fat/high-sucrose diet [23] induced changes in insulin sensitivity and body composition. Importantly, the beneficial systemic effects of hypertrophic skeletal muscle are due, in part, to multiple myokines and their effects on adipose tissue, bone, liver, and pancreas [15], thus demonstrating the importance of muscle as a secretory organ.

The C2C12 myogenic cell line has proven invaluable in furthering our understanding of the cellular and molecular processes underlying myogenesis. In conditions of low serum, subconfluent C2C12 myoblasts differentiate into skeletal myosin heavy chain expressing myocytes, which then fuse to form multinucleated myotubes with biochemical, biophysical, and immunohistological properties similar to mature skeletal muscle myofibers [31]. C2C12 myoblasts are amenable to plasmid-based gene transfer for gain-of-function studies, achieving transfection efficiencies of ˜33% [32]. However, transgene expression is diluted to levels below the western blotting detection threshold after 48 hours of differentiation [33], thus precluding functional assessment in mature myotubes. In contrast, multinucleated C2C12 myotubes are resistant to the introduction of plasmid DNA using traditional calcium-phosphate or liposomal transfection techniques, with only 1-5% of myotubes expressing the transgene [34]. In vitro transcribed synthetic mRNAs containing modified nucleotides have been shown to be efficiently transduced into multiple cells types both in vitro and in vivo [35], however the utilization of this method for the study of myonuclear protein regulation of paracrine signaling has not been previously explored.

In the C2C12 in vitro model of myogenesis, we observed Akt1 to transiently translocate into the nuclei of nascent maturing myotubes. Akt1 is known to be nuclear localized in multiple cancer cell lines [25, 26, 28], and to translocate into the nuclei of post-mitotic cardiomyocytes in response to anti-apoptotic atrial natriuretic peptide (ANP) stimulation [29]. Cofactors such as Tcl1 are known to mediate the nuclear localization of Akt1 [27, 45] as Akt1 lacks a known nuclear localization signal sequence. Tcl1 is an oncogene associated with T-cell leukemia, although related proteins with similar function may be expressed in nascent and mature myotubes. Importantly, cardiomyocyte specific transgenic overexpression of nuclear localized Akt1 inhibited myocyte apoptosis, reducing myocardial infarct size [46] and compensatory hypertrophy [47] in models of ischemia reperfusion injury and pressure overload, respectively. Importantly, ectopic nuclear localized Akt1 did not promote nuclear division or DNA synthesis [46], strongly suggesting that its anti-apoptotic and anti-hypertrophic effects on cardiomyocytes were mediated by kinase activity and subsequent changes in gene expression. The present observations of Akt1 translocation to the nucleus in maturing myotubes occur at a similar time point with the induction and/or increased expression of genes involved in muscle contractile function, cell cycle arrest, and resistance to apoptosis [39, 48-50].

Myogenesis is a complex multicellular process involving myogenic cells, tissue resident fibroblasts, and infiltrating immune cells. Damaged myofibers secrete multiple factors in order to both recruit monocytes/macrophages for the removal of cellular debris and to induce resident fibroblast secretion of the extracellular matrix proteins essential for muscle repair [51]. Multiple studies have indicated that chronic inflammation plays a causal role in promoting muscle fibrosis through a combination of excessive fibroblast activation and the prolonged residency of inflammatory cells [52]. Though the molecular mechanism is unclear, the present data indicate that ectopic myonuclear Akt1 impairs oxidative stress induced fibrogenic paracrine signaling. Without wishing to be bound by theory, these observations are consistent with the notion that myonuclear Akt1 is involved in modulating the paracrine secretory profile of maturing myotubes for the purposes of impairing fibroblast activation and/or inflammatory cell recruitment prior to these processes becoming deleterious. Importantly, though both the myotube and undifferentiated myoblast cell populations were both subject to oxidative stress, the nuclear localized Akt1 transgene was selectively expressed within myotubes.

Engineered Akt

The present results show that Akt1 is activated at the plasma membrane in response to insulin/IGF-1 stimulation. Activated Akt1 serves as a signaling integrator of multiple intra-and inter-cellular signaling pathways promoting growth, proliferation, and longevity. Akt1 was originally identified as an oncogene, promoting cell survival by bypassing apoptotic signaling pathways, and mutations of Akt1 (constitutively active) are known to be oncogenic. Importantly, the major site of biological activity in the cancer literature is in the nucleus, where it promotes the cell survival (oncogenic) phenotype through poorly understood mechanisms. To the best of the present inventors' knowledge, Akt1 has not been previously documented in the nuclei of skeletal muscle myocytes, and the method by which it is translocated into the nucleus of mitotic cells is unknown. Typically a gene has a nuclear localization sequence (NLS), which signals the cell to move the protein into the nucleus; however, native Akt1 does not have a known NLS.

Provided herein are genetically modified versions of Akt1 that include an exogenous nuclear localization sequence (NLS), e.g., at least one, two, three, or more NLS that force nuclear translocation of the modified Akt1. These are referred to herein as Akt1-NLS, or Akt1-NLS fusion proteins.

The following is an exemplary sequence of a sequence encoding an engineered mouse Akt1 comprising three NLS:

(SEQ ID NO: 1)
cagtGCGATCGCGGCGCGCCGCCACCATGAACGACGTAGCCATTGTGAA
GGAGGGCTGGCTGCACAAACGAGGGGAATATATTAAAACCTGGCGGCCA
CGCTACTTCCTCCTCAAGAACGATGGCACCTTTATTGGCTACAAGGAAC
GGCCTCAGGATGTGGATCAGCGAGAGTCCCCACTCAACAACTTCTCAGT
GGCACAATGCCAGCTGATGAAGACAGAGCGGCCAAGGCCCAACACCTTT
ATCATCCGCTGCCTGCAGTGGACCACAGTCATTGAGCGCACCTTCCATG
TGGAAACGCCTGAGGAGCGGGAAGAATGGGCCACCGCCATTCAGACTGT
GGCAGATGGACTCAAGAGGCAGGAAGAAGAGACGATGGACTTCCGATCA
GGCTCACCCAGTGACAACTCAGGGGCTGAAGAGATGGAGGTGTCCCTGG
CCAAGCCCAAGCACCGTGTGACCATGAACGAGTTTGAGTACCTGAAGCT
ACTGGGCAAGGGCACCTTTGGGAAGGTGATTCTGGTGAAAGAGAAGGCC
ACAGGCCGCTACTATGCCATGAAGATCCTCAAGAAGGAGGTCATCGTCG
CCAAGGATGAGGTTGCCCACACGCTTACTGAGAACCGTGTCCTGCAGAA
CTCTAGGCATCCCTTCCTTACGGCCCTCAAGTACTCATTCCAGACCCAC
GACCGCCTCTGCTTTGTCATGGAGTATGCCAACGGGGGCGAGCTCTTCT
TCCACCTGTCTCGAGAGCGTGTGTTCTCCGAGGACCGGGCCCGCTTCTA
TGGTGCGGAGATTGTGTCTGCCCTGGACTACTTGCACTCCGAGAAGAAC
GTGGTGTACCGGGACCTGAAGCTGGAGAACCTCATGCTGGACAAGGACG
GGCACATCAAGATAACGGACTTCGGGCTGTGCAAGGAGGGGATCAAGGA
CGGTGCCACTATGAAGACATTCTGCGGAACGCCGGAGTACCTGGCCCCT
GAGGTGCTGGAGGACAACGACTACGGCCGTGCAGTGGACTGGTGGGGGC
TGGGCGTGGTCATGTACGAGATGATGTGTGGCCGCCTGCCCTTCTACAA
CCAGGACCACGAGAAGCTGTTCGAGCTGATCCTCATGGAGGAGATCCGC
TTCCCGCGCACACTCGGCCCTGAGGCCAAGTCCCTGCTCTCCGGGCTGC
TCAAGAAGGACCCTACACAGAGGCTCGGTGGGGGCTCCGAGGATGCCAA
GGAGATCATGCAGCACCGGTTCTTTGCCAACATCGTGTGGCAGGATGTG
TATGAGAAGAAGCTGAGCCCACCTTTCAAGCCCCAGGTCACCTCTGAGA
CTGACACCAGGTATTTCGATGAGGAGTTCACAGCTCAGATGATCACCAT
CACGCCGCCTGATCAAGATGACAGCATGGAGTGTGTGGACAGTGAGCGG
AGGCCGCACTTCCCCCAGTTCTCCTACTCAGCCAGTGGCACAGCCGCGG
CCGCAGATCCAAAAAAGAAGAGAAAGGTAGATCCAAAAAAGAAGAGAAA
GGTAGATCCAAAAAAGAAGAGAAAGGTAGATACGGCCGCAGAACAAATG
GACTACAAAGACGATGACGATAAAATGGACTACAAAGACGATGACGATA
AATGAGGCGCGCCACGCGTcagt
Kozak-NT 21-26
Native Akt1-A²⁷ through C¹⁴⁶⁶
Linker1/3x-NLS/linker2/2x-FLAG-STOP-G¹⁴⁶⁷ through A¹⁶²²
Linker1-GCGGCCGCA (SEQ ID NO: 2)
3x-NLS-G¹⁴⁷⁶ through A¹⁵⁴⁷ (NLS: GATCCAAAAAAGAAGAGAAAGGTA; SEQ ID NO: 3)
Linker2-GATACGGCCGCAGAACAA (SEQ ID NO: 4)
2x-FLAG-STOP-A¹⁵⁶⁶ through A¹⁶²²
PH Domain-G³⁹-T³⁵⁰

The engineered Akt-NLS can include a sequence encoding all or an active part of native human Akt1 protein, e.g., encoding SEQ ID NO:5:

| (SEQ ID NO: 5) | |
|---|---|
| 1 | msdvaivkeg wlhkrgeyik twrpryfllk ndgtfigyke rpqdvdqrea plnnfsvaqc |
| 61 | qlmkterprp ntfiirclqw ttviertfhv etpeereewt taiqtvadgl kkqeeeemdf |
| 121 | rsgspsdnsg aeemevslak pkhrvtmnef eylkllgkgt fgkvilvkek atgryyamki |
| 181 | lkkevivakd evahtltenr vlqnsrhpfl talkysfqth drlcfvmeya nggelffhls |
| 241 | rervfsedra rfygaeivsa ldylhseknv vyrdlklenl mldkdghiki tdfglckegi |
| 301 | kdgatmktfc gtpeylapev ledndygrav dwwglgvvmy emmcgrlpfy nqdheklfel |
| 361 | ilmeeirfpr tlgpeaksll sgllkkdpkg rlgggsedak eimqhrffag ivwqhvyekk |
| 421 | lsppfkpqvt setdtryfde eftaqmitit ppdqddsmec vdserrphfp qfsysasgta. |

In some embodiments, the PH domain (amino acids 4-111 of SEQ ID NO:5) is deleted. In some embodiments, the PH domain and a linker (amino acids 4-129 or 5-129 of SEQ ID NO:5) is deleted. In some embodiments, the sequence comprises amino acids 112-479 or 130-479 of SEQ ID NO:5.

An exemplary sequence encoding human Akt1 is shown in NCBI GenBank Sequence ID NM_005163.2 (SEQ ID NO:6), e.g., a sequence comprising nucleotides 575 to 1978, or 555-1997, of SEQ ID NO:6:

| (SEQ ID NO: 6) | |
|---|---|
| 1 | taattatggg tctgtaacca ccctggactg ggtgctcctc actgacggac ttgtctgaac |
| 61 | ctctctttgt ctccagcgcc cagcactggg cctggcaaaa cctgagacgc ccggtacatg |
| 121 | ttggccaaat gaatgaacca gattcagacc ggcaggggcg ctgtggttta ggaggggcct |
| 181 | ggggtttctc ccaggaggtt tttgggcttg cgctggaggg ctctggactc ccgtttgcgc |
| 241 | cagtggcctg catcctggtc ctgtcttcct catgtttgaa tttctttgct ttcctagtct |
| 301 | ggggagcagg gaggagccct gtgccctgtc ccaggatcca tgggtaggaa caccatggac |
| 361 | agggagagca aacggggcca tctgtcacca ggggcttagg gaaggccgag ccagcctggg |
| 421 | tcaaagaagt caaaggggct gcctggagga ggcagcctgt cagctggtgc atcagaggct |
| 481 | gtggccaggc cagctgggct cggggagcgc cagcctgaga ggagcgcgtg agcgtcgcgg |
| 541 | gagcctcggg caccatgagc gacgtggcta ttgtgaagga gggttggctg cacaaacgag |
| 601 | gggagtacat caagacctgg cggccacgct acttcctcct caagaatgat ggcaccttca |
| 661 | ttggctacaa ggagcggccg caggatgtgg accaacgtga ggctcccctc aacaacttct |
| 721 | ctgtggcgca gtgccagctg atgaagacgg agcggccccg gcccaacacc ttcatcatcc |
| 781 | gctgcctgca gtggaccact gtcatcgaac gcaccttcca tgtggagact cctgaggagc |
| 841 | gggaggagtg gacaaccgcc atccagactg tggctgacgg cctcaagaag caggaggagg |
| 901 | aggagatgga cttccggtcg ggctcaccca gtgacaactc aggggctgaa gagatggagg |
| 961 | tgtccctggc caagcccaag caccgcgtga ccatgaacga gtttgagtac ctgaagctgc |
| 1021 | tgggcaaggg cactttcggc aaggtgatcc tggtgaagga gaaggccaca ggccgctact |
| 1081 | acgccatgaa gatcctcaag aaggaagtca tcgtggccaa ggacgaggtg gcccacacac |
| 1141 | tcaccgagaa ccgcgtcctg cagaactcca ggcacccctt cctcacagcc ctgaagtact |
| 1201 | ctttccagac ccacgaccgc ctctgctttg tcatggagta cgccaacggg ggcgagctgt |
| 1261 | tcttccacct gtcccgggag cgtgtgttct ccgaggaccg ggcccgcttc tatggcgctg |
| 1321 | agattgtgtc agccctggac tacctgcact cggagaagaa cgtggtgtac cgggacctca |
| 1381 | agctggagaa cctcatgctg gacaaggacg ggcacattaa gatcacagac ttcgggctgt |
| 1441 | gcaaggaggg gatcaaggac ggtgccacca tgaagacctt ttgcggcaca cctgagtacc |
| 1501 | tggcccccga ggtgctggag gacaatgact acggccgtgc agtggactgg tgggggctgg |
| 1561 | gcgtggtcat gtacgagatg atgtgcggtc gcctgccctt ctacaaccag gaccatgaga |
| 1621 | agctttttga gctcatcctc atggaggaga tccgcttccc gcgcacgctt ggtcccgagg |
| 1681 | ccaagtcctt gctttcaggg ctgctcaaga aggaccccaa gcagaggctt ggcgggggct |
| 1741 | ccgaggacgc caaggagatc atgcagcatc gcttctttgc cggtatcgtg tggcagcacg |
| 1801 | tgtacgagaa gaagctcagc ccacccttca agccccaggt cacgtcggag actgacacca |
| 1861 | ggtattttga tgaggagttc acggcccaga tgatcaccat cacaccacct gaccaagatg |
| 1921 | acagcatgga gtgtgtggac agcgagcgca ggccccactt cccccagttc tcctactcgg |
| 1981 | ccagcggcac ggcctgaggc ggcggtggac tgcgctggac gatagcttgg agggatggag |
| 2041 | aggcggcctc gtgccatgat ctgtatttaa tggtttttat ttctcgggtg catttgagag |
| 2101 | aagccacgct gtcctctcga gcccagatgg aaagacgttt ttgtgctgtg ggcagcaccc |
| 2161 | tcccccgcag cggggtaggg aagaaaacta tcctgcgggt tttaatttat ttcatccagt |
| 2221 | ttgttctccg ggtgtggcct cagccctcag aacaatccga ttcacgtagg gaaatgttaa |
| 2281 | ggacttctgc agctatgcgc aatgtggcat tggggggccg ggcaggtcct gcccatgtgt |
| 2341 | cccctcactc tgtcagccag ccgccctggg ctgtctgtca ccagctatct gtcatctctc |
| 2401 | tggggccctg ggcctcagtt caacctggtg gcaccagatg caacctcact atggtatgct |
| 2461 | ggccagcacc ctctcctggg ggtggcaggc acacagcagc cccccagcac taaggccgtg |
| 2521 | tctctgagga cgtcatcgga ggctgggccc ctgggatggg accagggatg ggggatgggc |
| 2581 | cagggtttac ccagtgggac agaggagcaa ggtttaaatt tgttattgtg tattatgttg |
| 2641 | ttcaaatgca ttttgggggt ttttaatctt tgtgacagga aagccctccc ccttcccctt |
| 2701 | ctgtgtcaca gttcttggtg actgtcccac cgggagcctc cccctcagat gatctctcca |
| 2761 | cggtagcact tgaccttttc gacgcttaac ctttccgctg tcgccccagg ccctccctga |
| 2821 | ctccctgtgg gggtggccat ccctgggccc ctccacgcct cctggccaga cgctgccgct |
| 2881 | gccgctgcac cacggcgttt ttttacaaca ttcaacttta gtatttttac tattataata |
| 2941 | taatatggaa ccttccctcc aaattcttca ataaaagttg cttttcaaaa aaaaaaaaaa |
| 3001 | aaaaaaaa |

An exemplary mouse Akt1 sequence is available in GenBank at NP_033782.1, and is shown in SEQ ID NO:7:

| (SEQ ID NO: 7) | |
|---|---|
| 1 | mndvaivkeg wlhkrgeyik twrpryfllk ndgtfigyke rpqdvdqres plnnfsvaqc |
| 61 | qlmkterprp ntfiirclqw ttviertfhv etpeereewa taiqtvadgl krqeeetmdf |
| 121 | rsgspsdnsg aeemevslak pkhrvtmnef eylkllgkgt fgkvilvkek atgryyamki |
| 181 | lkkevivakd evahtltenr vlqnsrhpfl talkysfqth drlcfvmeya nggelffhls |
| 241 | rervfsedra rfygaeivsa ldylhseknv vyrdlklenl mldkdghiki tdfglckegi |
| 301 | kdgatmktfc gtpeylapev ledndygrav dwwglgvvmy emmcgrlpfy nqdheklfel |
| 361 | ilmeeirfpr tlgpeaksll sgllkkdptq rlgggsedak eimqhrffan ivwqdvyekk |
| 421 | lsppfkpqvt setdtryfde eftaqmitit ppdqddsmec vdserrphfp qfsysasgta |

In some embodiments, the PH domain (amino acids 5-105, or 6-104, of SEQ ID NO:7) is deleted. In some embodiments, the PH domain and a linker (amino acids 4-129 or 5-129 of SEQ ID NO:7) is deleted. In some embodiments, the sequence comprises amino acids 105-479 or 130-479 of SEQ ID NO:7.

An exemplary nucleic acid sequence encoding a mouse Akt1 is available in GenBank as NM_009652.3 (SEQ ID NO:8), i.e., nucleotides 371-1813 of SEQ ID NO:8.

| (SEQ ID NO: 8) | |
|---|---|
| 1 | gcggggcggg gagaaggcgg gccggcggcg gcggcggcag caccgagtcg gcgggcggcc |
| 61 | ggcccagcgc ggcagcgcac gcgagtccgg gaccagcgga gcggaccgag cagcgtcctg |
| 121 | tggccggcac cgcggcggcc cagatccggc cagcagcgcg cgcccggacg ccgctgcctt |
| 181 | cagccggccc cgcccagcgc ccgcccgcgg gatgcggagc ggcgggcgcc cgaggccgcg |
| 241 | gcccggctag gcccagtcgc ccgcacgcgg cggcccgacg ctgcggccag gccggctggg |
| 301 | ctcagcctac cgagaagaga ctctgagcat catccctggg ttacccctgt ctctgggggc |
| 361 | cacggatacc atgaacgacg tagccattgt gaaggagggc tggctgcaca aacgagggga |
| 421 | atatattaaa acctggcggc cacgctactt cctcctcaag aacgatggca cctttattgg |
| 481 | ctacaaggaa cggcctcagg atgtggatca gcgagagtcc ccactcaaca acttctcagt |
| 541 | ggcacaatgc cagctgatga agacagagcg gccaaggccc aacaccttta tcatccgctg |
| 601 | cctgcagtgg accacagtca ttgagcgcac cttccatgtg gaaacgcctg aggagcggga |
| 661 | agaatgggcc accgccattc agactgtggc agatggactc aagaggcagg aagaagagac |
| 721 | gatggacttc cgatcaggct cacccagtga caactcaggg gctgaagaga tggaggtgtc |
| 781 | cctggccaag cccaagcacc gtgtgaccat gaacgagttt gagtacctga agctactggg |
| 841 | caagggcacc tttgggaagg tgattctggt gaaagagaag gccacaggcc gctactatgc |
| 901 | catgaagatc ctcaagaagg aggtcatcgt cgccaaggat gaggttgccc acacgcttac |
| 961 | tgagaaccgt gtcctgcaga actctaggca tcccttcctt acggccctca agtactcatt |
| 1021 | ccagacccac gaccgcctct gctttgtcat ggagtatgcc aacgggggcg agctcttctt |
| 1081 | ccacctgtct cgagagcgtg tgttctccga ggaccgggcc cgcttctatg gtgcggagat |
| 1141 | tgtgtctgcc ctggactact tgcactccga gaagaacgtg gtgtaccggg acctgaagct |
| 1201 | ggagaacctc atgctggaca aggacgggca catcaagata acggacttcg ggctgtgcaa |
| 1261 | ggaggggatc aaggacggtg ccactatgaa gacattctgc ggaacgccgg agtacctggc |
| 1321 | ccctgaggtg ctggaggaca acgactacgg ccgtgcagtg gactggtggg ggctgggcgt |
| 1381 | ggtcatgtac gagatgatgt gtggccgcct gcccttctac aaccaggacc acgagaagct |
| 1441 | gttcgagctg atcctcatgg aggagatccg cttcccgcgc acactcggcc ctgaggccaa |
| 1501 | gtccctgctc tccgggctgc tcaagaagga ccctacacag aggctcggtg ggggctccga |
| 1561 | ggatgccaag gagatcatgc agcaccggtt ctttgccaac atcgtgtggc aggatgtgta |
| 1621 | tgagaagaag ctgagcccac ctttcaagcc ccaggtcacc tctgagactg acaccaggta |
| 1681 | tttcgatgag gagttcacag ctcagatgat caccatcacg ccgcctgatc aagatgacag |
| 1741 | catggagtgt gtggacagtg agcggaggcc gcacttcccc cagttctcct actcagccag |
| 1801 | tggcacagcc tgaggcctgg ggcagcggct ggcagctcca cgctcctctg cattgccgag |
| 1861 | tccagaagcc ccgcatggat catctgaacc tgatgttttg tttctcggat gcgctgggga |
| 1921 | ggaaccttgc cagcctccag gaccagggga ggatgtttct actgtgggca gcagcctacc |
| 1981 | tcccagccag gtcaggagga aaactatcct ggggtttttc ttaatttatt tcatccagtt |
| 2041 | tgagaccaca catgtggcct cagtgcccag aacaattaga ttcatgtaga aaactattaa |
| 2101 | ggactgacgc gaccatgtgc aatgtgggct catgggtctg ggtgggtccc gtcactgccc |
| 2161 | ccattggcct gtccaccctg gccgccacct gtctctaggg tccagggcca aagtccagca |
| 2221 | agaaggcacc agaagcaccc ccctgtggta tgctaactgg ccctctccct ctgggcgggg |
| 2281 | agaggtcaca gctgcttcag ccctagggct ggatgggatg gccagggctc aagtgaggtt |
| 2341 | gacagaggaa caagaatcca gtttgttgct gtgtcccatg ctgttcagag acatttaggg |
| 2401 | gattttaatc ttggtgacag gagagcccct gccctcccgc acccgctccc gcgtggtggc |
| 2461 | tcttagcggg taccctggga gcgcctgcct cacgtgagcc cttctcctag cacttgtcct |
| 2521 | tttagatgct ttccctctcc cgctgtccgt caccctggcc tgtcccctcc cggccagacg |
| 2581 | ctggccattg ctgcaccatg tcgtttttta caacattcag cttcagcatt tttactatta |
| 2641 | taataagaaa ctgtccctcc aaattcaata aaaattgctt ttcaagcttg aaaaaaaaaa |
| 2701 | aaaaaaa |

The proteins include at least one nuclear localization sequence, i.e., one or more short sequences of positively charged lysines or arginines exposed on the protein surface, e.g., SV40 large T antigen NLS (PKKKRRV (SEQ ID NO:9)) or nucleoplasmin NLS (KRPAATKKAGQAKKKK (SEQ ID NO:10)) or DPKKKRKV (SEQ ID NO:11). Other NLSs are known in the art; see, e.g., Cokol et al., EMBO Rep. 2000 Nov. 15; 1(5): 411-415; Freitas and Cunha, Curr Genomics. 2009 December; 10(8): 550-557. In some embodiments, two, three, four, or more NLS are included; the NLS can be all the same or can be different. The NLS can be included at the N terminus, C terminus, or internally within the protein, so long as activity of the Akt1 is retained, e.g., the ability to impair apoptosis in mitotic cells or post-mitotic cells that can undergo apoptosis, e.g., cardiac myocytes, or to impair oxidative stress-induced pro-fibrotic paracrine signaling.

In some embodiments, the components of the fusion proteins are at least 80%, e.g., at least 85%, 90%, 95%, 97%, or 99% identical to the amino acid sequence of a exemplary sequence (e.g., as provided herein), e.g., have differences at up to 1%, 2%, 5%, 10%, 15%, or 20% of the residues of the exemplary sequence replaced, e.g., with conservative mutations, e.g., including or in addition to the alterations described herein. In preferred embodiments, the variant retains a desired activity of the parent, e.g., the ability to impair apoptosis in mitotic cells or post-mitotic cells that can undergo apoptosis, e.g., cardiac myocytes; or the ability to impair oxidative stress-induced pro-fibrotic paracrine signaling, e.g., in the C2C12 model.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). For purposes of the present compositions and methods, at least 80% of the full length of the sequence is aligned.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Also provided herein are isolated nucleic acids encoding the Akt-NLS fusion proteins, vectors comprising the isolated nucleic acids, optionally operably linked to one or more regulatory domains for expressing the Akt-NLS proteins, and host cells, e.g., mammalian host cells, comprising the nucleic acids, and optionally expressing the Akt-NLS proteins. In some embodiments, the host cells are stem cells or progenitor cells, or are myocytes, e.g., skeletal muscle cells. Also provided herein are transgenic animals, e.g., mice comprising one or more sequences encoding an Akt-NLS protein as described herein, e.g., stably integrated into the genome of some or all of the cells of the animal, e.g., in their germ cells.

Expression Systems

To use the Akt-NLS fusion proteins described herein, it may be desirable to express them from a nucleic acid that encodes them. This can be performed in a variety of ways. For example, the nucleic acid encoding the Akt-NLS fusion can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the Akt-NLS fusion for production of the Akt-NLS fusion protein. The nucleic acid encoding the Akt-NLS fusion protein can also be cloned into an expression vector, for administration to a plant cell, animal cell, preferably a mammalian cell or a human cell, fungal cell, bacterial cell, or protozoan cell.

To obtain expression, a sequence encoding a Akt-NLS fusion protein is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of a nucleic acid depends on the particular application. For example, a strong constitutive promoter is typically used for expression and purification of fusion proteins. In contrast, when the Akt-NLS fusion protein is to be administered in vivo, a promoter that restrict expression to muscle cells, e.g., to skeletal muscle cells, can be used.

Exemplary promoters include the following:

SkM specific promoters:

HSA—Human skeletal actin (e.g., as described in Miniou et al., Nucleic Acids Res. 1999 Oct. 1; 27(19): e27)

MCK enhancer/dMCK/tMCK/enh358MCK (and other shortened modification of the MCK promoter) (e.g., as described in Wang et al., Gene Therapy (2008) 15, 1489-1499)

Striated muscle (includes both skeletal and cardiac muscle) promoters:

MCK—muscle creatine kinase (e.g., as described in Bruning et al., Molecular Cell, Vol. 2,559-569 (1998))

In addition, a preferred promoter for administration of the Akt-NLS fusion protein can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the Akt-NLS fusion protein, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into a cell or is selected with regard to the intended use of the Akt-NLS fusion protein, e.g., expression in intact or isolated cells from plants, animals, bacteria, fungus, protozoa, etc. Standard bacterial expression vectors include plasmids such as pBR322 based plasmids, pSKF, pET23D, and commercially available tag-fusion expression systems such as GST and LacZ, as well as viral vectors.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins, e.g., under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

In some embodiments, the plasmid is pR26_CAG_AsiSI/MluI.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the gRNA encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the Akt-NLS fusion protein.

Viral Vectors

Viral vectors for use in the present methods and compositions include recombinant retroviruses, adenovirus, adeno-associated virus, alphavirus, and lentivirus.

A preferred viral vector system useful for delivery of nucleic acids in the present methods is the adeno-associated virus (AAV). AAV is a tiny non-enveloped virus having a 25 nm capsid. No disease is known or has been shown to be associated with the wild type virus. AAV has a single-stranded DNA (ssDNA) genome. AAV has been shown to exhibit long-term episomal transgene expression, and AAV has demonstrated excellent transgene expression in the brain, particularly in neurons. Vectors containing as little as 300 base pairs of AAV can be packaged and can integrate. Space for exogenous DNA is limited to about 4.7 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. For example, AAV9 has been shown to efficiently cross the blood-brain barrier. Moreover, the AAV capsid can be genetically engineered to increase transduction efficient and selectivity, e.g., biotinylated AAV vectors, directed molecular evolution, self-complementary AAV genomes and so on. In some embodiments, AAV9 is used. AAV serotype-9 (AAV9) has been shown to be highly specific for the delivery of transgenes to the heart and skeletal muscle, including diaphragm, with minimal infectivity of other organs such as lung and liver (Bish et al., Hum Gene Ther 19, 1359-1368 (2008); Zincarelli et al., Mol Ther 16, 1073-1080 (2008)). In some embodiments, AAV9 is used to deliver Akt1-NLS under control of a dMCK promoter, which has been previously demonstrated to drive the expression of a LacZ transgene specifically in skeletal muscle, including the diaphragm (Wang et al., Gene Ther 15, 1489-1499 (2008)).

Alternatively, retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Alphaviruses can also be used. Alphaviruses are enveloped single stranded RNA viruses that have a broad host range, and when used in gene therapy protocols alphaviruses can provide high-level transient gene expression. Exemplary alphaviruses include the Semliki Forest virus (SFV), Sindbis virus (SIN) and Venezuelan Equine Encephalitis (VEE) virus, all of which have been genetically engineered to provide efficient replication-deficient and -competent expression vectors. Alphaviruses exhibit significant neurotropism, and so are useful for CNS-related diseases. See, e.g., Lundstrom, Viruses. 2009 June; 1(1): 13-25; Lundstrom, Viruses. 2014 June; 6(6): 2392-2415; Lundstrom, Curr Gene Ther. 2001 May; 1(1):19-29; Rayner et al., Rev Med Virol. 2002 September-October; 12(5):279-96.

Non-viral methods can also be employed to cause expression of an Akt1-NLS as described herein. Typically non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol. 116(1):131-135 (2001); Cohen et al., Gene Ther. 7(22):1896-905 (2000); or Tam et al., Gene Ther. 7(21):1867-74 (2000).

In some embodiments, modified synthetic RNAs encoding the Akt-NLS proteins are used. The nucleic acid sequences encoding an Akt-NLS fusion protein as described herein can be synthesized in vitro by well-known chemical synthesis techniques, as described in, e.g., Adams, J. Am. Chem. Soc. 105:661, 1983; Belousov, Nucleic Acids Res. 25:3440-3444, 1997; Frenkel, Free Radic. Biol. Med. 19:373-380, 1995; Blommers, Biochemistry 33:7886-7896, 1994; Narang, Meth. Enzymol. 68:90, 1979; Brown, Meth. Enzymol. 68:109, 1979; Beaucage, Tetra. Lett. 22:1859, 1981; and U.S. Pat. No. 4,458,066.

Nucleic acid sequences (e.g., mRNA) comprising a sequence encoding an Akt-NLS fusion protein as described herein can be stabilized against nucleolytic degradation, nuclease stability, decrease the likelihood of triggering an innate immune response, lower the incidence of off-target effects, and/or improve pharmacodynamics relative to non-modified molecules so as to increase potency and specificity, such as by the incorporation of a modification, e.g., a nucleotide modification. For example, nucleic acid sequences encoding an Akt-NLS fusion protein as described herein can include a phosphorothioate, boranophosphate, or 4′-thio-ribose.

As another example, the nucleic acid sequence (e.g., mRNA) can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), 2′-O-(2′-methoxyethyl), 2′-O-alkyl, 2′-O-alkyl-O-alkyl, 2′-amino, 2′-deoxy-2′-fluoro-b-D-arabinonucleic acid. As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., nucleic acid analogues in which the ribose ring is “locked” by a methylene bridge connecting the 2′-O atom and the 4′-C atom (see, e.g., Kaupinnen et al., Drug Disc. Today 2(3):287-290, 2005; Koshkin et al., J. Am. Chem. Soc. 120(50):13252-13253, 1998). In some embodiments, the RNA is modified by pseudouridine and/or 5-methylcytidine substitution. For additional modifications see Kaczmarek et al., Genome Med. 2017; 9: 60, US 2010/0004320, US 2009/0298916, and US 2009/0143326.

The nucleic acid (e.g., mRNA) can be further modified at the 5′ end by capping the end, which is known in the art. At the end of transcription, the 5′ end of the RNA transcript contains a free triphosphate group since it was the first incorporated nucleotide in the chain. Capping replaces the triphosphate group with another structure called the “5′ cap”. Suitable 5′ caps include methylated guanosine. In some embodiments, a N7-methyl guanosine is connected to the 5′ nucleotide through a 5′ to 5′ triphosphate linkage, typically referred to as m7G cap, m7Gppp, or cap 0 in the literature. An additional methylation on the 2′O position of the initiating nucleotide generates Cap 1, or referred to as m7GpppNm-, where Nm denotes any nucleotide with a 2′O methylation. See, e.g., Kaczmarek et al., Genome Med. 2017; 9: 60.

In some embodiments, the nucleic acid (e.g., mRNA) is delivered using a liposome or nanoparticle, e.g., by attachment to or encapsulation within a biocompatible nanoparticle. The nanoparticles can be tagged with antibodies against cell surface antigens of the target tissue. In some embodiments, the nucleic acid is modified to with a N-acetylgalactosamine GalNAc-conjugate approach (include wherever lipid nanoparticle is mentioned). In some embodiments, the nucleic acid is conjugated with PEI or an antibody targeted to the tunica intima or other relevant cell types.

Examples of biocompatible nanoparticles include liposomes and polymeric nanoparticles. As used herein, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged nucleic acid molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

Polymeric nanoparticles for use in the present methods and compositions can comprise cationic polymers, such as amine-containing polymers, poly-L-lysine, polyamidoamine, and polyethyleneimine, chitosan, poly(β-amino esters). In some embodiments the cationic polymers electrostatically condense the negatively charged RNA into nanoparticles. See, e.g., Pack et al., Nat Rev Drug Discov. 2005 July; 4(7):581-93; Kaczmarek et al., Genome Med. 2017; 9: 60.

Techniques for the manipulation of nucleic acids used to practice this invention, such as, e.g., subcloning, labeling probes (e.g., random-primer labeling using Klenow polymerase, nick translation, and amplification), sequencing, hybridization, and the like are well described in the scientific and patent literature, see, e.g., Sambrook et al., Molecular Cloning; A Laboratory Manual 3d ed., 2001; Current Protocols in Molecular Biology, Ausubel et al., Eds. (John Wiley & Sons, Inc., New York, 2010); Kriegler, Gene Transfer and Expression: A Laboratory Manual, 1990; Laboratory Techniques in Biochemistry and Molecular Biology: Hybridization with Nucleic Acid Probes, Part I. Theory and Nucleic Acid Preparation, Tijssen, Ed., Elsevier, N.Y., 1993.

The present invention also includes nucleic acids (including DNA and RNA) encoding Akt1-NLS, Akt1-NLS proteins, vectors comprising the nucleic acids, cells comprising the vectors, as well as kits comprising the proteins and nucleic acids described herein, e.g., for use in a method described herein.

Transgenic Animals

Also provided herein are non-human transgenic animals. Such animals are useful, e.g., for studying the function and/or activity of nuclear-localised Akt1 protein. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. As used herein, a transgene is exogenous DNA encoding Akt1-NLS that preferably is integrated into or occurs in the genome of the cells of a transgenic animal, e.g., integrated into a Rosa26 locus, e.g., using CRISPR/Cas9, e.g., with the pR26 Asi targeting vector as described in Chu et al., BMC Biotechnology (2016) 16:4. In some embodiments, a transgenic animal can be one in which an endogenous Akt1 gene has been altered by, e.g., by homologous recombination between the endogenous gene and an exogenous DNA molecule introduced into a cell of the animal, e.g., an embryonic cell of the animal, prior to development of the animal, that adds one or more NLS to at least one allele of endogenous Akt1.

Intronic sequences and polyadenylation signals can also be included in the transgene to increase the efficiency of expression of the transgene. A tissue-specific regulatory sequence(s) can be operably linked to a transgene of the invention to direct expression of Akt1-NLS protein to particular cells. A transgenic founder animal can be identified based upon the presence of a Akt1-NLS transgene in its genome and/or expression of Akt1-NLS mRNA in tissues or cells of the animals. A transgenic founder animal can then be used to breed additional animals carrying the transgene. Moreover, transgenic animals carrying a transgene encoding a Akt1-NLS protein can further be bred to other transgenic animals carrying other transgenes.

Akt1-NLS proteins or polypeptides can be expressed in transgenic animals or plants, e.g., a nucleic acid encoding the protein or polypeptide can be introduced into the genome of an animal. In some embodiments, the nucleic acid is placed under the control of a tissue specific promoter, e.g., a muscle specific promoter, e.g., a skeletal or cardiac muscle specific promoter. Suitable animals are mice, rats, rabbist, pigs, cows, goats, and sheep. In some embodiments, the mice are generated using a conditional Cre-Lox “knockout” system, in which Cre-Lox recombination technology is used to achieve targeted deletion or expression of the Akt1-NLS transgene. For example, a promoter, e.g., a CAG or tissue-specific promoter, is inactivated by the presence of a Floxed Puromycin/Stop (Puro/Stop) cassette placed between the promoter and the transgene start site/ATG, thereby preventing transcription of the transgene. Transgene expression under the control of the promoter is accomplished by CRE mediated recombination, removing the Puro/Stop cassette, thereby allowing transcription of the transgene. In some embodiments, the mice are crossed with an mouse having a drug-inducible and/or tissue specific expression of CRE, e.g., a tamoxifen inducible Mer-Cre-Mer (MCM), driven by skeletal or striated muscle specific promoter, e.g., a human skeletal actin promoter.

The invention also includes a population of cells from a transgenic animal.

In some embodiments, the transgenic animals are made by crossing an animal having an Akt1-NLS integrated into their genome with another animal, e.g., with a murine X-linked muscular dystrophy (mdx) (Duchenne's Muscular Dystrophy model, e.g., as described in Stedman et al., Nature. 1991 Aug. 8; 352(6335):536-9); mice deficient in the heart/muscle isoform of the adenine nucleotide translocator (Anti) (mitochondrial/metabolic myopathy model, as described in Graham et al., Nat Genet. 1997 July; 16(3):226-34.); or other animal model of disease, e.g., MDX2CV, MDX3CV, MDX4CV, MDX5CV, or MDX^{4CV}/mTR^{G2} mice, which are humanized and more severe models of the MDX mouse line (see, e.g., Im et al., Human Molecular Genetics, 1996, Vol. 5, No. 8 1149-1153; Beastrom et al., The American Journal of Pathology, Vol. 179, No. 5, November 2011; Chang et al., Proc Natl Acad Sci USA. 2016 Nov. 15; 113(46):13120-13125).

Methods of Use

The Akt-NLS fusion proteins and nucleic acids as described herein can be used to promote cellular resiliency and adaptability to stress, which in turn impairs the secretion of factors that promote immune cell infiltration and the activation of fibroblasts to secrete extra cellular matrix proteins leading to fibrotic scar tissue.

The methods described herein include methods for the treatment of disorders associated with fibrosis. Generally, the methods include administering a therapeutically effective amount of an Akt-NLS fusion protein or nucleic acid as described herein, to a subject who is in need of, or who has been determined to be in need of, such treatment.

As used in this context, to “treat” means to ameliorate at least one symptom of the disorder associated with insterstitial muscle fibrosis. Often, muscle fibrosis results in progressive muscle wasting and weakness; thus, a treatment (e.g., administration of a therapeutically effective amount of a compound described herein) can result in a reduction in muscle wasting and weakness, or a reduction in rate of progression of wasting and weakness.

Duchenne Muscular Dystrophy (DMD) is an X-linked recessive disorder caused by a non-sense mutation in the dystrophin gene (DMD), resulting in a premature truncation of the dystrophin protein. DMD patients have progressive muscle wasting and weakness, often progressing to wheelchair dependency by the end of the first decade, assisted ventilation by the end of the second decade, with premature death in the second to fourth decades (Guiraud et al., Annu Rev Genomics Hum Genet 16, 281-308 (2015)). At the histological level, muscles from DMD patients show disorganized myofibers, high levels of infiltrated inflammatory cells, and extensive deposits of adipose and fibrotic tissue (Rahimov and Kunkel, J Cell Biol 201, 499-510 (2013)). A longitudinal study in DMD patients showed a strong correlation between the extent of interstitial fibrosis and functional decline (Desguerre et al., J. Neuropathol Exp Neurol 68, 762-773 (2009)), while TGF-β1 blockade promoted apoptosis of Lin-α7-Sca-1+ cells 45 impairing the progression of interstitial fibrosis and functional decline in the mdx mouse model of Duchenne's (Taniguti et al., Muscle Nerve 43, 82-87 (2011)).

Thus provided herein is a method to reduce muscle fibrosis in patients with Duchenne Muscular Dystrophy (DMD), e.g., in the diaphragm, heart, and limb skeletal muscles of subjects with DMD.

The present methods can also be used in trauma patients who have experienced volumetric muscle loss; and in surgical patients in whom incisions through muscle fascia often result in extensive fibrosis, thus impairing functional recovery. In addition, the present methods can be used in subjects who have other conditions associated with increased immune cell content and interstitial fibrosis in muscles, e.g., in striated muscle, e.g., in skeletal or cardiac muscle, including mitochondrial/metabolic myopathies; idiopathic inflammatory myopathy; myocardial remodeling/hypertrophy/heart failure; and other myopathies in which inflammation and fibrosis (extracellular remodeling) play a causal role in the disease etiology.

The methods can include delivering modified synthetic mRNA encoding the nuclear localized Akt1 directly to myocytes, e.g., via local injection or infusion into a muscle. Modified synthetic mRNA can be used to induce protein expression that is transient (e.g., lasting up to 4 days) with no risk of genomic integration. Alternatively, the methods can include using a viral vector, e.g., an adeno-associated virus, for systemic and local delivery of nuclear localized Akt1 specifically into skeletal or cardiac muscle myocytes in vivo, e.g., a viral vector that includes a skeletal and/or cardiac muscle-specific promoter.

### EXAMPLES

The invention is further described in the following examples, which do not limit the scope of the invention described in the claims.

### Example 1. Nuclear Localized Akt1 Limits Skeletal Muscle Derived Fibrotic Signaling

Materials and Methods

The following materials and methods were used in Example 1.

Cell culture. C2C12 and 10T1/2 cells were grown in Dulbecco's Modified Eagle's medium supplemented with 10% fetal bovine serum. C2C12 at ˜75% confluency were induced to differentiate by switching the medium to differentiation medium (DMEM supplemented with 2% horse serum).

Cellular Fractionation. C2C12 cells were lifted from the cell culture plastic using trypsin-EDTA and pelleted by centrifugation at 15,000×g for 5 minutes. The pellet was resuspended in 100 μl of Cytoplasmic Extraction Buffer (10 mM HEPES, 60 mM KCl, 1 mM EDTA, 1% NP40, adjusted to pH 7.4) supplemented with protease and phosphatase inhibitors. The cell-buffer mixture was incubated on ice for 30 minutes, centrifuged at 1000×g for 10 minutes at 4° C.; the supernatant was collected as the cytoplasmic fraction. The pellet was washed with PBS 3 times and resuspended in 40 μl of Nuclear Extraction Buffer (20 mM TrisHCl, 420 mM NaCl, 1.5 mM MgCl, 0.2 mM EDTA, adjusted to pH 8.0) supplemented with protease and phosphatase inhibitors. The mixture was incubated on ice for 15 minutes, centrifuged at 15,000×g for 10 minutes at 4° C., after which the supernatant was collected as the nuclear fraction.

Immunoblotting. Protein content was determined by DC protein assay (Bio-Rad, Hercules, Calif.) with known concentrations of BSA as standards. Protein concentrations were equalized by the addition of an appropriate volume of lysis buffer to ensure equal loading of 30 μg of either total, cytoplasmic, or nuclear proteins. Primary antibodies were visualized with HRP-conjugated goat anti-rabbit and anti-mouse antibodies.

Immunostaining. Differentiated myotubes were immunostained and visualized according to previously described methods [36]. Briefly, C2C12 myotubes were fixed with 4% PFA, permeabilized with 0.1% Triton X-100 in PBS, and blocked in 5% donkey serum. Cells were then incubated with mouse anti-myosin heavy chain (MF-20, Developmental Studies Hybridoma Bank) and rabbit anti-GFP (ab183734, AbCam). Primary antibodies were visualized with Alexa-Flour 594 conjugated donkey anti-mouse and Alexa-Flour 488 conjugated donkey anti-rabbit antibodies. Coverslips were mounted using Fluoromount-G (Southern Biotech, Birmingham AL).

Image Processing. Acquired images were minimally process in Photoshop (Adobe). Contrast was enhanced with auto-contrast function on the whole image file. Nuclei were false colored and overlaid onto the MF20 images with either the screen or color function.

In vitro transcription (IVT) of modified mRNA and transfection. Template generation and IVT of modified mRNA was performed as previously described [37]. Briefly, the IVT template was generated by first linearizing the pcDNA3.1(+) plasmid containing either Akt1 (NM_009652) or GFP by restriction digestion with Xba1. A 3× nuclear localization sequence (NLS) was inserted immediately prior to the stop codon. The linearized plasmid was PCR-amplified using both a CMV forward primer and a custom-designed reverse primer incorporating a Poly(T) tail of 120 thymidines using Kapa HiFi HotStart PCR ReadyMix (Kapa Biosystems, Wilmington, Mass.). Following QIAquick PCR purification, the tailed PCR product was used as the template for IVT reactions performed with the MEGAscript T7 kit (Ambion by Life Technologies, Carlsbad, Calif.), supplemented with 6 mM 3′-O-Me-m7G(5′)ppp(5′)G ARCA cap analog (New England Biolabs, Ipswich, Mass.), 1.5 mM GTP, 7.5 mM ATP (MEGAscript T7 kit), 7.5 mM 5-methyl CTP and 7.5 mM pseudo UTP (TriLink Biotechnologies, San Diego, Calif.). Completed IVT reactions were treated by TURBO DNAse for 15 min, Antarctic Phosphatase for 30 min, followed by purification using the MegaClear kit. The final mRNA product was quantified by spectrophotometry, diluted to 100 ng/μl in elution buffer, and stored at −80° C. until use. Cells in 6-well plates were transfected with 1 μg of modified RNAs containing using Lipofectamine MessengerMAX.

Conditioned media treatment. C2C12 cells were seeded in six well plates and differentiated at 75% confluency by adding DMEM supplemented with 2% horse serum. The cells were transfected on day 4 of differentiation with either mod-AKT-NLS or mod-GFP-NLS and treated with 250 μM hydrogen peroxide 4 hours after transfection. Non-transfected and non-treated cells were included as controls. Twenty-four (24) hours after treatment, the media was collected and centrifuged at 1000×g for 10 min at 4° C., and the supernatant was mixed with an equal volume of fresh media (DMEM+2% FBS) for further use as conditioned media. 10T1/2 cells were seeded in six well plates and treated with unconditioned fresh or conditioned media upon reaching ˜75% confluency; cells were harvested after 24 hours in TRIzol for RNA extraction.

Quantitative real-time PCR. Total RNA was extracted with TRIzol reagent (Life Technologies, #15-596-018) and cDNA was synthesized from 2 μg of total RNA using M-MLV Reverse Transcriptase (Invitrogen, #28025-013). Quantitative real-time PCR was performed using Power SYBR Green Master Mix (Applied Biosystems). All data were normalized to Hprt as the internal control.

Statistical Analysis. Results were expressed as mean±standard deviation and differences were determined by Student's t test. The level of significance was set at p≤0.05.

Primers:

Primer FW:
(SEQ ID NO: 12)
cagtGCGATCGCGGCGCGCCGCCACCATGAACGACGTAGCCATTGT
Primer REV:
(SEQ ID NO: 13)
actgACGCGTGGCGCGCCTCATTTATCGTCATCGTCTTTGTAGTCCATT
TTATCGTCATCGTCTTTGTAGTCCATTTGTTCTGCGGCCGTATC
Sequencing Primers:
AKT1seq-FW 1 = CGACGTAGCCATTGTGAAGG (SEQ ID NO: 14)
AKT1seq-REV1 = ACACCTCCATCTCTTCAGCC (SEQ ID NO: 15)
AKT1seq-FW2 = GGCTGAAGAGATGGAGGTGT (SEQ ID NO: 16)
AKT1seq-REV2 = CGTTCTTCTCGGAGTGCAAG (SEQ ID NO: 17)
AKT1seq-FW3 = GCTGCTCAAGAAGGACCCTA (SEQ ID NO: 18)
AKT1seq-REV3 = TAGTCCATTTGTTCTGCGGC (SEQ ID NO: 19)

### Example 1A. Akt1 is Transiently Nuclear Localized During Myogenesis

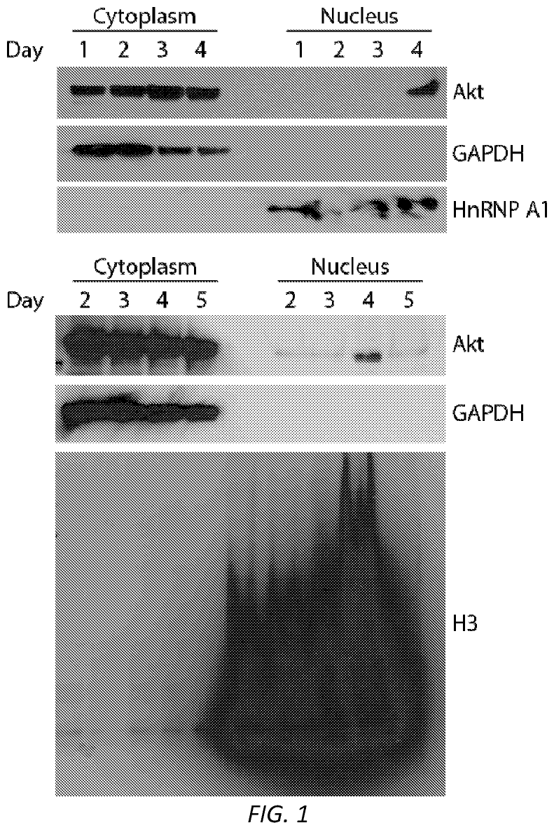

We asked whether nuclear Akt1 signaling occurs during myogenesis. To address this, we performed nuclear-cytoplasmic fractionation of C2C12 cells during the 5-day time course of differentiation into myotubes. We observe Akt1 to be expressed throughout differentiation (FIG. 1) and consistent with prior reports indicating a consistent level of Akt1 expression in the whole homogenates of differentiating C2C12 cells [38, 39]. Interestingly, we observe Akt1 to transiently accumulate in the nucleus at day-4 (D4) of differentiation (FIG. 1), suggestive of a specific temporal-dependent role during myogenesis.

### Example 1B. Synthetic Modified RNA is an Efficient Means for Gene Delivery to Myotubes

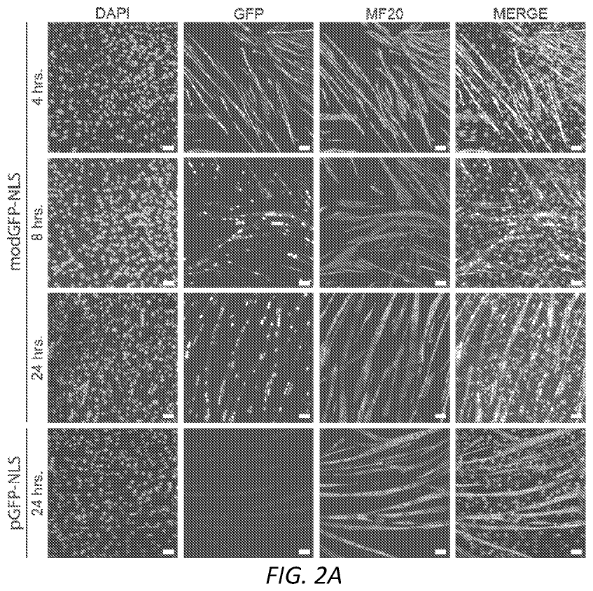

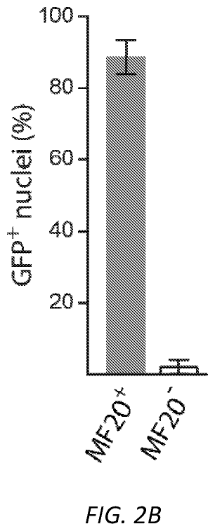

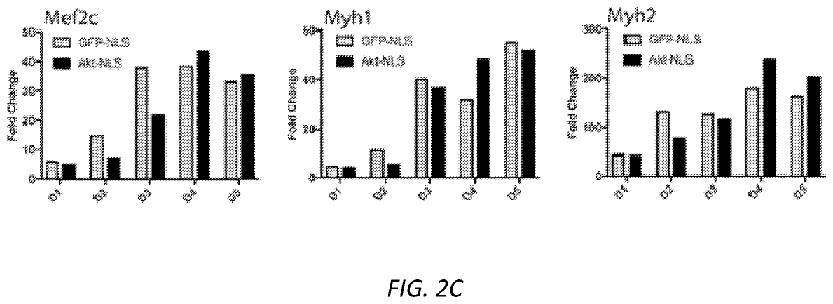

To explore the biological role of nuclear localized Akt, we transfected C2C12 myoblasts at D4 of differentiation with a pcDNA3.1 plasmid containing Akt1 or GFP with a 3× nuclear localization signal on its 3′ end. Immunofluorescent images of myotubes 24-hours after transfection indicate no GFP⁺ nuclei (FIG. 2A), either in myotubes or in unfused myoblasts. We next tried synthetic modified RNA as a delivery vehicle as it has been previously shown to have high transfection efficiency in cultured hESCs [40] and in murine cardiomyocytes in vivo [41]. Using our existing plasmids, we generated synthetic modified RNAs encoding nuclear localized Akt1 and GFP and transfected C2C12 myoblasts at D4 of differentiation. Immunofluorescent images 24-hours after transfection indicated robust GFP expression (FIG. 2A) in ˜85% of myonuclei (FIG. 2B) as defined by DAPI stain being within an MF20⁺ myotube. Importantly, nearly all MF20⁻ cells are GFP⁻ (<2% of MF20⁻ cells are GFP⁺) suggesting that the synthetic modRNAs are preferentially transduced into multinucleated myotubes formed as compared to MF20⁻ myoblasts.

To further characterize this technique for ectopic gene expression in differentiated myotubes, we next characterized the temporal expression of ectopic proteins following modRNA transfection. In D4 myotubes transfected with modGFP-NLS, we observed ectopic GFP expression as early as 4 hours after transfection, with robust expression and nuclear localization by 8 hours (FIG. 2A). These observations are in agreement with prior reports indicating a rapid and transient protein expression following modRNA treatment [41, 42]. Together these observations indicate that modRNA is a more effective vehicle for transient gene delivery to mature myotubes in culture than plasmids.

Western blot analysis of modAkt1-NLS-FLAG treated C2C12 myotubes indicate detectable levels of nuclear FLAG-tagged Akt1 as early as 2 hours post transfection (data not shown), while preliminary studies indicate that modAkt1-NLS-FLAG treatment of myoblasts does not impair the expression of myogenic regulatory factors or terminal differentiation markers (FIG. 2C). Together, these observations indicate that nuclear Akt1 has a role both independent of myoblast/myocyte differentiation status and specific to maturing myotubes.

### Example 1C. Nuclear-Localized Akt1 Impairs Fibrogenic Paracrine Signaling

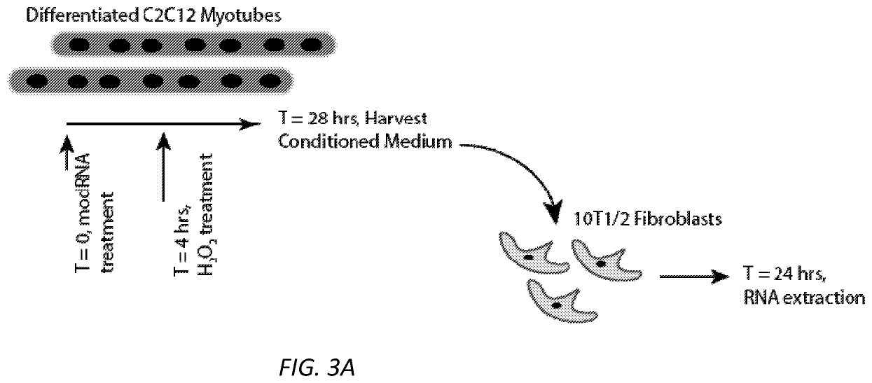

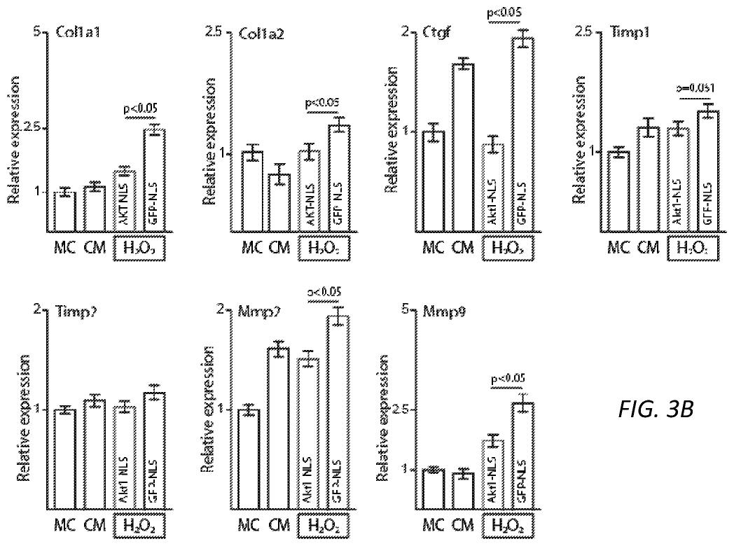

To elucidate the biological role of myonuclear Akt1 within the context of the heterocellular skeletal muscle tissue, we decided to assay for changes in myotube derived paracrine signaling under conditions of stress. We utilized a conditioned medium approach in which the medium of D4 myotubes was conditioned for 24-hours under conditions of oxidative stress following the transfection with either modAkt1-NLS or modGFP-NLS. Myotubes were treated with 250 μM H₂O₂ to induce non-lethal oxidative stress. 10T1/2 cells treated with the conditioned media for 24-hours and then were assayed for expression changes of genes associated with fibrosis (FIG. 3A). 10T1/2 cells were treated with unconditioned C2C12 differentiation medium (Medium Control, MC), and 24-hour conditioned C2C12 differentiation medium (Conditioned Medium, CM), as controls. Apart from Ctgf, Timp1, and Mmp2 (FIG. 3B), quantitative PCR (qPCR) analyses indicate that 10T1/2 cells treated with CM express similar levels of fibrosis associated genes as 10T1/2 cells treated with MC. Importantly, the conditioned medium from modGFP-NLS treated myotubes under conditions of oxidative stress consistently induce a greater increase in fibrosis associated gene expression as compared to the conditioned medium from modAkt-NLS treated myotubes with oxidative stress (FIG. 3B). Together, these observations indicate that ectopic nuclear localized Akt1 can impair oxidative stress induced fibrotic paracrine signaling.

### Example 2. In Vivo Validation—Akt1-NLS Mice

We cloned in a modified form of full length murine Akt1 into the pR26 CAG AsiSI/MluI cloning vector (Chu V T, et al. BMC Biotechnology 2016). The pR26 cloning vector utilized the CAG promoter to drive expression of transgenes inserted into the AsiSI/MluI restriction sites. The CAG promoter is inactivated by the presence of a Floxed Puromycin/Stop (Puro/Stop) cassette placed between the CAG promoter and the AsiSI/MluI restriction sites, thereby preventing transcription of the transgene. Transgene expression under the control of the CAG promoter is accomplished by CRE mediated recombination, removing the Puro/Stop cassette, thereby allowing transcription of the transgene. CRISPR/Cas9 was utilized to target the Rosa26 locus in mouse embryos according to previously described methodologies (Chu V T, et al. BMC Biotechnology 2016).

The modified full length Akt1 consists of a KOZAC sequence immediately upstream of the Akt1 start codon, and the replacement of the Akt1 stop codon with a 3×-NLS/2×-FLAG-STOP cassette. This cassette contains a 3× nuclear localization sequence (NLS), followed by a short linker region, a 2× FLAG cassette, and an in frame stop codon. Thus, the Akt1-NLS-FLAG-StOP cassette inserted into the pR26 CAG AsiSI/MluI vector encodes for a longer and nuclear targeted version of Akt1 than the native genome-encoded Akt1. Mice positive for this transgene are hereafter referred to as Rosa26^{Akt1-NLS/+} (hemizygous for transgene) or Rosa26^{Akt1-NLS/Akt1-NLS} (homozygous for transgene) mice.

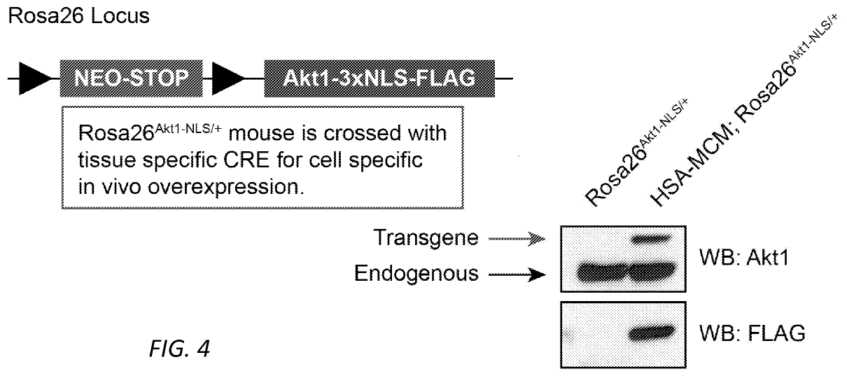

Rosa26^{Akt1-NLS/Akt1-NLS} mice were crossed with a tamoxifen inducible Mer-Cre-Mer (MCM) driven by the human skeletal actin promoter, hereafter the HSA-MCM line to generate HSA-MCM; Rosa26^{Akt1-NLS/+} and Rosa26^{Akt1-NLS/+} mice. Both mice were injected with tamoxifen for 5 consecutive days to activate the Cre recombinase, and tissues were harvested 3 days after the last injection for subsequent western blotting for transgene expression. FIG. 4 shows the results of Western blotting experiments to validate the expression of the transgene (WB for Akt1 detected both endogenous and transgene protein, WB for FLAG detects transgene protein). Note that the transgenic Akt1 was only observed in the CRE+ mouse.

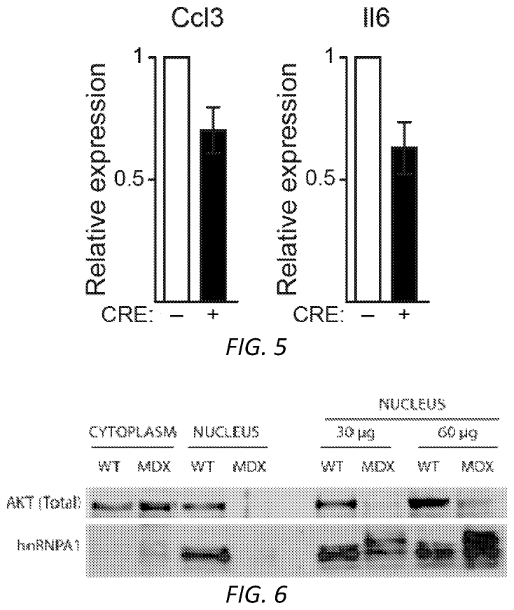

Pilot qPCR was performed on gastrocnemius muscles obtained from n=2 Akt-NLS and HSA-MCM; Akt-NLS mice following tamoxifen treatment described above. The results, shown in FIG. 5, demonstrated a decrease in expression of IL-6 and CCL3.

### Example 3: The Role of Enhanced Myonuclear Akt1 Signaling in the Regenerative and Fibrogenic Capacity of Aged Skeletal Muscle

The mdx mouse is the most widely used Duchenne Muscular Dystrophy (DMD) laboratory model, recapitulating many of DMD symptoms including elevated levels of creatine kinase, moderate myopathy marked by early necrosis, and the progressive degeneration, fibrosis, and functional impairment of the diaphragm (Guiraud et al., Annu Rev Genomics Hum Genet 16, 281-308 (2015); Stedman et al., Nature. 1991 Aug. 8; 352(6335):536-9). We quantified the myonuclear content of skeletal muscle under normal physiological conditions as a function of age in both WT and mdx mice. The age-dependent myonuclear content was assayed in WT (n=5 male and n=5 female) and mdx mice (n=5) at 3, 9, and 12 months of age. Only male mdx mice were assayed given the nature of X-linked neuromuscular disorders only affecting males, with females typically being non-syndromic carriers. Additional WT mice at 24-months mice are similarly assayed. Gastrocnemius muscle wet weight was normalized to tibial length and subsequently fixed in 4% PFA and prepared for cryo-sectioning. Both vastus medialis muscles were isolated, weighed, and subjected to myonuclear isolation as previously described (Ohkawa et al., Methods Mol Biol 798, 517-530 (2012)). Briefly, freshly isolated skeletal muscles were treated with collagenase II separating individual myofibers from the mononuclear fraction. Following mincing in hypotonic buffer, the nuclei were isolated by ultracentrifugation on a sucrose gradient. The total nuclei content was split into thirds, ⅓ for isolation of genomic DNA for nuclei quantification, with the remaining ⅔ used for protein extraction and subsequent western blot analyses. Genomic DNA (gDNA) content was quantified by qPCR using chromatin immunoprecipitation (ChIP) validated GAPDH primers from a linear curve generated from gDNA/cell quantity standards (Active Motive, Carlsbad, Calif.). Westerns were quantified by densitometry using ImageJ software and the intensities were normalized to total nuclei number. The results showed that Myonuclear Akt1 content was nearly absent in the muscles of 9-month old mdx mice (FIG. 6).

AAV9 is used to deliver Akt1-NLS-FLAG under control of the dMCK promoter, which has been previously demonstrated to drive the expression of a LacZ transgene specifically in skeletal muscle, including the diaphragm (Wang et al., Gene Ther 15, 1489-1499 (2008)). Using WT mice, we assay for the effects of AAV9-dMCK-Akt1-NLS-FLAG (AAV9-Akt1-NLS) treatment on the muscle tissue microenvironment; specifically, fibrogenic activity and MuSC mediated muscle repair.

Effect of ectopic nuclear Akt1 on aged and mdx phenotypes—AAV9-Akt1-NLS (1×10¹¹-10¹² vector genomes, vgs) is administered intravenously into WT male mice (n=8) at 3, 9, and 18 months of age, and into mdx mice (n=8) at 3-months of age, as previously described^{127,130,131}. Only male mice are examined as there are no known gender-based differences in either AAV infectivity or muscle regeneration reported. Mice injected with AAV9-GFP-NLS will be used as negative controls. Two (2) months post AAV9 injection, WT mice are subjected to cryoinjury of the tibialis anterior (TA) muscle according to methodologies established within the laboratory¹³². Thirty (30) days post injury, mice are euthanized for excision of the injured and uninjured contralateral control TA muscles. One (1) and 6-months post AAV treated mdx mice are exercised on a programmable treadmill with adjustable incline; time to exhaustion, maximum distance, and work performed will be measured using well established methodologies^{67,133-135}. Following the 9-month functional assessment, the mice are euthanized and the diaphragm, tibialis anterior, and gastrocnemius muscles are excised for histological and qPCR analyses.

Histological and qPCR analyses—Excised muscles are be fixed in 4% PFA, sectioned, and stained with Hematoxylin & Eosin (H&E) and Picrosirius Red for assessment of overall tissue morphology and the extent of interstitial fibrosis. Images are quantified using ImageJ and CellProfiler software as previously described (Neppl et al., J Cell Biol 216, 3497-3507 (2017); Jones et al., BMC Bioinformatics 9, 482 (2008)). Quantitative PCR (qPCR) analyses are performed on the excised tissues from AAV9-Akt1-NLS and AAV9-GFP-NLS treated mice to examine the effects of ectopic nuclear Akt1 on fibrogenic signaling. Specifically, we focus on markers for ECM deposition and maturation (e.g. Col1a1/2, Lox, loxl1, etc.), fibrogenic paracrine factors (e.g. TGFβ1, CTGF, TNFα, etc.), and activated fibroblasts (e.g. PDGFRα, Postn, αSMA, etc.).

### REFERENCES

[1] H. M. Blau, B. D. Cosgrove, A. T. Ho, The central role of muscle stem cells in regenerative failure with aging, Nat Med, 21 (2015) 854-862.

[2] J. M. Haus, J. A. Carrithers, S. W. Trappe, T. A. Trappe, Collagen, cross-linking, and advanced glycation end products in aging human skeletal muscle, J Appl Physiol (1985), 103 (2007) 2068-2076.

[3] M. Visser, B. H. Goodpaster, S. B. Kritchevsky, A. B. Newman, M. Nevitt, S. M. Rubin, E. M. Simonsick, T. B. Harris, Muscle mass, muscle strength, and muscle fat infiltration as predictors of incident mobility limitations in well-functioning older persons, J Gerontol A Biol Sci Med Sci, 60 (2005) 324-333.

[4] J. D. Walston, Sarcopenia in older adults, Curr Opin Rheumatol, 24 (2012) 623-627.

[5] S. J. Meng, L. J. Yu, Oxidative stress, molecular inflammation and sarcopenia, Int J Mol Sci, 11 (2010) 1509-1526.

[6] H. Yin, F. Price, M. A. Rudnicki, Satellite cells and the muscle stem cell niche, Physiological reviews, 93 (2013) 23-67.

[7] F. S. Tedesco, A. Dellavalle, J. Diaz-Manera, G. Messina, G. Cossu, Repairing skeletal muscle: regenerative potential of skeletal muscle stem cells, The Journal of clinical investigation, 120 (2010) 11-19.

[8] R. L. Neppl, M. Kataoka, D. Z. Wang, Crystallin-alphaB Regulates Skeletal Muscle Homeostasis via Modulation of Argonaute2 Activity, J Biol Chem, 289 (2014) 17240-17248.

[9] A. S. Brack, T. A. Rando, Intrinsic changes and extrinsic influences of myogenic stem cell function during aging, Stem Cell Rev, 3 (2007) 226-237.

[10] A. S. Brack, M. J. Conboy, S. Roy, M. Lee, C. J. Kuo, C. Keller, T. A. Rando, Increased Wnt signaling during aging alters muscle stem cell fate and increases fibrosis, Science, 317 (2007) 807-810.

[11] I. M. Conboy, M. J. Conboy, A. J. Wagers, E. R. Girma, I. L. Weissman, T. A. Rando, Rejuvenation of aged progenitor cells by exposure to a young systemic environment, Nature, 433 (2005) 760-764.

[12] R. I. Sherwood, J. L. Christensen, I. L. Weissman, A. J. Wagers, Determinants of skeletal muscle contributions from circulating cells, bone marrow cells, and hematopoietic stem cells, Stem cells, 22 (2004) 1292-1304.

[13] M. Sinha, Y. C. Jang, J. Oh, D. Khong, E. Y. Wu, R. Manohar, C. Miller, S. G. Regalado, F. S. Loffredo, J. R. Pancoast, M. F. Hirshman, J. Lebowitz, J. L. Shadrach, M. Cerletti, M. J. Kim, T. Serwold, L. J. Goodyear, B. Rosner, R. T. Lee, A. J. Wagers, Restoring systemic GDF11 levels reverses age-related dysfunction in mouse skeletal muscle, Science, 344 (2014) 649-652.

[14] J. V. Chakkalakal, K. M. Jones, M. A. Basson, A. S. Brack, The aged niche disrupts muscle stem cell quiescence, Nature, 490 (2012) 355-360.

[15] B. K. Pedersen, M. A. Febbraio, Muscles, exercise and obesity: skeletal muscle as a secretory organ, Nature reviews. Endocrinology, 8 (2012) 457-465.

[16] M. M. Reza, N. Subramaniyam, C. M. Sim, X. Ge, D. Sathiakumar, C. McFarlane, M. Sharma, R. Kambadur, Irisin is a pro-myogenic factor that induces skeletal muscle hypertrophy and rescues denervation-induced atrophy, Nat Commun, 8 (2017) 1104.

[17] L. Zeng, Y. Akasaki, K. Sato, N. Ouchi, Y. Izumiya, K. Walsh, Insulin-like 6 is induced by muscle injury and functions as a regenerative factor, J Biol Chem, 285 (2010) 36060-36069.

[18] C. Zhang, Y. Li, Y. Wu, L. Wang, X. Wang, J. Du, Interleukin-6/signal transducer and activator of transcription 3 (STAT3) pathway is essential for macrophage infiltration and myoblast proliferation during muscle regeneration, J Biol Chem, 288 (2013) 1489-1499.

[19] G. Song, G. Ouyang, S. Bao, The activation of Akt/PKB signaling pathway and cell survival, J Cell Mol Med, 9 (2005) 59-71.

[20] D. J. Glass, Signaling pathways that mediate skeletal muscle hypertrophy and atrophy, Nat Cell Biol, 5 (2003) 87-90.

[21] M. Laplante, D. M. Sabatini, mTOR signaling in growth control and disease, Cell, 149 (2012) 274-293.

[22] A. Musaro, K. McCullagh, A. Paul, L. Houghton, G. Dobrowolny, M. Molinaro, E. R. Barton, H. L. Sweeney, N. Rosenthal, Localized Igf-1 transgene expression sustains hypertrophy and regeneration in senescent skeletal muscle, Nat Genet, 27 (2001) 195-200.

[23] Y. Izumiya, T. Hopkins, C. Morris, K. Sato, L. Zeng, J. Viereck, J. A. Hamilton, N. Ouchi, N. K. LeBrasseur, K. Walsh, Fast/Glycolytic muscle fiber growth reduces fat mass and improves metabolic parameters in obese mice, Cell Metab, 7 (2008) 159-172.

[24] Y. Akasaki, N. Ouchi, Y. Izumiya, B. L. Bernardo, N. K. Lebrasseur, K. Walsh, Glycolytic fast-twitch muscle fiber restoration counters adverse age-related changes in body composition and metabolism, Aging Cell, 13 (2014) 80-91.

[25] R. Wang, M. G. Brattain, AKT can be activated in the nucleus, Cell Signal, 18 (2006) 1722-1731.

[26] A. M. Martelli, G. Tabellini, D. Bressanin, A. Ognibene, K. Goto, L. Cocco, C. Evangelisti, The emerging multiple roles of nuclear Akt, Biochim Biophys Acta, 1823 (2012) 2168-2178.

[27] Y. Pekarsky, A. Koval, C. Hallas, R. Bichi, M. Tresini, S. Malstrom, G. Russo, P. Tsichlis, C. M. Croce, Tcl1 enhances Akt kinase activity and mediates its nuclear translocation, Proc Natl Acad Sci USA, 97 (2000) 3028-3033.

[28] J. J. Wallin, J. Guan, W. W. Prior, K. A. Edgar, R. Kassees, D. Sampath, M. Belvin, L. S. Friedman, Nuclear phospho-Akt increase predicts synergy of PI3K inhibition and doxorubicin in breast and ovarian cancer, Sci Transl Med, 2 (2010) 48ra66.

[29] T. Kato, J. Muraski, Y. Chen, Y. Tsujita, J. Wall, C. C. Glembotski, E. Schaefer, M. Beckerle, M. A. Sussman, Atrial natriuretic peptide promotes cardiomyocyte survival by cGMP-dependent nuclear accumulation of zyxin and Akt, J Clin Invest, 115 (2005) 2716-2730.

[30] J. A. Muraski, M. Rota, Y. Misao, J. Fransioli, C. Cottage, N. Gude, G. Esposito, F. Delucchi, M. Arcarese, R. Alvarez, S. Siddiqi, G. N. Emmanuel, W. Wu, K. Fischer, J. J. Martindale, C. C. Glembotski, A. Leri, J. Kajstura, N. Magnuson, A. Berns, R. M. Beretta, S. R. Houser, E. M. Schaefer, P. Anversa, M. A. Sussman, Pim-1 regulates cardiomyocyte survival downstream of Akt, Nat Med, 13 (2007) 1467-1475.

[31] D. K. McMahon, P. A. Anderson, R. Nassar, J. B. Bunting, Z. Saba, A. E. Oakeley, N. N. Malouf, C2C12 cells: biophysical, biochemical, and immunocytochemical properties, Am J Physiol, 266 (1994) C1795-1802.

[32] E. Dodds, M. G. Dunckley, K. Naujoks, U. Michaelis, G. Dickson, Lipofection of cultured mouse muscle cells: a direct comparison of Lipofectamine and DOSPER, Gene Ther, 5 (1998) 542-551.

[33] S. E. Mercer, D. Z. Ewton, X. Deng, S. Lim, T. R. Mazur, E. Friedman, Mirk/Dyrk1B mediates survival during the differentiation of C2C12 myoblasts, J Biol Chem, 280 (2005) 25788-25801.

[34] C. Antolik, P. G. De Deyne, R. J. Bloch, Biolistic transfection of cultured myotubes, Sci STKE, 2003 (2003) PL11.

[35] K. R. Chien, L. Zangi, K. O. Lui, Synthetic chemically modified mRNA (modRNA): toward a new technology platform for cardiovascular biology and medicine, Cold Spring Harb Perspect Med, 5 (2014) a014035.

[36] R. L. Neppl, C. L. Wu, K. Walsh, lncRNA Chronos is an aging-induced inhibitor of muscle hypertrophy, J Cell Biol, 216 (2017) 3497-3507.

[37] P. K. Mandal, D. J. Rossi, Reprogramming human fibroblasts to pluripotency using modified mRNA, Nat Protoc, 8 (2013) 568-582.

[38] H. Ren, D. Accili, C. Duan, Hypoxia converts the myogenic action of insulin-like growth factors into mitogenic action by differentially regulating multiple signaling pathways, Proc Natl Acad Sci USA, 107 (2010) 5857-5862.

[39] Y. Fujio, K. Guo, T. Mano, Y. Mitsuuchi, J. R. Testa, K. Walsh, Cell cycle withdrawal promotes myogenic induction of Akt, a positive modulator of myocyte survival, Mol Cell Biol, 19 (1999) 5073-5082.

[40] T. Akiyama, S. Sato, N. Chikazawa-Nohtomi, A. Soma, H. Kimura, S. Wakabayashi, S. B. H. Ko, M. S. H. Ko, Efficient differentiation of human pluripotent stem cells into skeletal muscle cells by combining RNA-based MYOD1-expression and POU5F1-silencing, Sci Rep, 8 (2018) 1189.

[41] L. Zangi, K. O. Lui, A. von Gise, Q. Ma, W. Ebina, L. M. Ptaszek, D. Spater, H. Xu, M. Tabebordbar, R. Gorbatov, B. Sena, M. Nahrendorf, D. M. Briscoe, R. A. Li, A. J. Wagers, D. J. Rossi, W. T. Pu, K. R. Chien, Modified mRNA directs the fate of heart progenitor cells and induces vascular regeneration after myocardial infarction, Nat Biotechnol, 31 (2013) 898-907.

[42] L. Warren, P. D. Manos, T. Ahfeldt, Y. H. Loh, H. Li, F. Lau, W. Ebina, P. K. Mandal, Z. D. Smith, A. Meissner, G. Q. Daley, A. S. Brack, J. J. Collins, C. Cowan, T. M. Schlaeger, D. J. Rossi, Highly efficient reprogramming to pluripotency and directed differentiation of human cells with synthetic modified mRNA, Cell Stem Cell, 7 (2010) 618-630.

[43] B. D. Manning, L. C. Cantley, AKT/PKB signaling: navigating downstream, Cell, 129 (2007) 1261-1274.

[44] K. M. Lai, M. Gonzalez, W. T. Poueymirou, W. O. Kline, E. Na, E. Zlotchenko, T. N. Stitt, A. N. Economides, G. D. Yancopoulos, D. J. Glass, Conditional activation of akt in adult skeletal muscle induces rapid hypertrophy, Mol Cell Biol, 24 (2004) 9295-9304.

[45] J. Laine, G. Kunstle, T. Obata, M. Sha, M. Noguchi, The protooncogene TCL1 is an Akt kinase coactivator, Mol Cell, 6 (2000) 395-407.

[46] I. Shiraishi, J. Melendez, Y. Ahn, M. Skavdahl, E. Murphy, S. Welch, E. Schaefer, K. Walsh, A. Rosenzweig, D. Torella, D. Nurzynska, J. Kajstura, A. Leri, P. Anversa, M. A. Sussman, Nuclear targeting of Akt enhances kinase activity and survival of cardiomyocytes, Circ Res, 94 (2004) 884-891.

[47] Y. Tsujita, J. Muraski, I. Shiraishi, T. Kato, J. Kajstura, P. Anversa, M. A. Sussman, Nuclear targeting of Akt antagonizes aspects of cardiomyocyte hypertrophy, Proc Natl Acad Sci USA, 103 (2006) 11946-11951.

[48] J. Wang, K. Walsh, Resistance to apoptosis conferred by Cdk inhibitors during myocyte differentiation, Science, 273 (1996) 359-361.

[49] J. F. Chen, E. M. Mandel, J. M. Thomson, Q. Wu, T. E. Callis, S. M. Hammond, F. L. Conlon, D. Z. Wang, The role of microRNA-1 and microRNA-133 in skeletal muscle proliferation and differentiation, Nat Genet, 38 (2006) 228-233.

[50] A. Blais, C. J. van Oevelen, R. Margueron, D. Acosta-Alvear, B. D. Dynlacht, Retinoblastoma tumor suppressor protein-dependent methylation of histone H3 lysine 27 is associated with irreversible cell cycle exit, J Cell Biol, 179 (2007) 1399-1412.

[51] S. B. Charge, M. A. Rudnicki, Cellular and molecular regulation of muscle regeneration, Physiological reviews, 84 (2004) 209-238.

[52] C. J. Mann, E. Perdiguero, Y. Kharraz, S. Aguilar, P. Pessina, A. L. Serrano, P. Munoz-Canoves, Aberrant repair and fibrosis development in skeletal muscle, Skelet Muscle, 1 (2011) 21.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated engineered protein comprising an AKT serine/threonine kinase 1 (Akt1) linked to at least one nuclear localization sequence (NLS), optionally with a linker sequence therebetween.

2. The isolated engineered protein of claim 1, wherein the Akt1 comprises mouse or human Akt1.

3. The isolated engineered protein of claim 2, wherein the mouse Akt1 sequence is at least 80% identical to SEQ ID NO:7.

4. The isolated engineered protein of claim 2, wherein the human Akt1 sequence is at least 80% identical to SEQ ID NO:5.

5. The isolated engineered protein of claim 1, wherein the NLS comprises SV40 large T antigen NLS (PKKKRRV (SEQ ID NO:9)); nucleoplasmin NLS (KRPAATKKAGQAKKKK (SEQ ID NO:10); or DPKKKRKV (SEQ ID NO:11).

6. An isolated nucleic acid encoding the isolated engineered protein of claim 1.

7. A vector comprising the isolated nucleic acid of claim 6.

8. The vector of claim 6, which is a plasmid vector or viral vector.

9. The vector of claim 8, which is an adeno-associated virus (AAV) vector.

10. The vector of claim 9, which is an AAV serotype-9 (AAV9).

11. The isolated nucleic acid of claim 6, which is a modified synthetic RNA.

12. The isolated nucleic acid of claim 11, which is modified to include a 5′ cap and/or a 3′ polyadenylation sequence.

13. An isolated cell comprising the isolated nucleic acid of claim 6, optionally expressing the isolated engineered protein of claim 1.

14. A transgenic mouse, wherein one or more cells of the mouse comprise a sequence encoding an AKT serine/threonine kinase 1 (Akt1) linked to at least one nuclear localization sequence (NLS), optionally with a linker sequence therebetween, integrated in to the genome of the cell.

15. An isolated cell or tissue from the transgenic mouse of claim 14.

16. A method of reducing muscle fibrosis in a subject, the method comprising administering to muscle tissue of the subject a protein comprising an AKT serine/threonine kinase 1 (Akt1) linked to at least one nuclear localization sequence (NLS), optionally with a linker sequence therebetween.

17. A method of reducing muscle fibrosis in a subject, the method comprising administering a nucleic acid encoding a protein comprising an AKT serine/threonine kinase 1 (Akt1) linked to at least one nuclear localization sequence (NLS), optionally with a linker sequence therebetween, preferably wherein the nucleic acid comprises a promoter that directs expression specifically in striated muscle cells of the subject.

18. The method of claim 17, wherein the promoter directs expression specifically in skeletal muscle cells of the subject.

19. The method of claim 17, wherein the nucleic acid comprises a viral vector.

20. The method of claim 19, wherein the viral vector is an adeno-associated virus (AAV) vector.

21. The method of claim 20, wherein the AAV vector is an AAV serotype-9 (AAV9).

22. The method of claim 17, wherein the nucleic acid comprises a modified synthetic RNA.

23. The method of claim 22, wherein the nucleic acid is modified to include a 5′ cap and/or a 3′ polyadenylation sequence.

24. The method of claim 17, wherein the subject has muscular dystrophy, is a trauma patient who has experienced volumetric muscle loss; is a surgical patient in whom incisions through muscle fascia have resulted in extensive fibrosis; has a mitochondrial/metabolic myopathy; has an idiopathic inflammatory myopathy; has myocardial remodeling/hypertrophy/heart failure.
